(19) United States
(12) Patent Application Publication
(10) Pub. No.: US 2002/0086001 A1
(43) Pub. Date: Jul. 4, 2002

(54) Normalization of defective T cell responsiveness through manipulation of thymic regeneration

(76) Inventor: Richard Boyd, Prahran (AU)

Correspondence Address: IRELL & MANELLA LLP, 1800 AVENUE OF THE STARS, SUITE 900, LOS ANGELES, CA, 90067, US

(21) Appl. No.: 09/976,599

(22) Filed: Oct. 12, 2001

(30) Foreign Application Priority Data
Apr. 17, 2000 (AU) PCT/AU/00329
Apr. 15, 1999 (AU) PP9778
Oct. 13, 2000 (AU) PRO745

(51) Int. Cl. A61K 48/00; A61K 38/08; A61K 38/10

(52) U.S. Cl. 424/93.21; 424/93.7; 514/15

(57) ABSTRACT

The present disclosure provides methods for the treatment and potential alleviation of autoimmune diseases and allergies in a patient. This is accomplished by deleting at least most of the existing T cell population and reactivating the thymus. Optionally, hematopoietic stem cells, autologous, syngeneic, allogeneic or xenogeneic, are delivered to increase the speed of regeneration of the patient's immune system and to supply normal T cells to the patient or to replace existing aberrant T cells. In a preferred embodiment the hematopoietic stem cells are CD34+. The patient's thymus is reactivated by disruption of sex steroid mediated signaling to the thymus. In a preferred embodiment, this disruption is created by administration of LHRH agonists, LHRH antagonists, anti-LHRH receptor antibodies, anti-LHRH vaccines or combinations thereof.

This application is a continuation-in-part of U.S. Ser. No. not yet available filed Sep. 26, 2001, which is a continuation-in-part of U.S. Ser. No. 09/755,983, filed Jan. 5, 2001, which is a continuation-in-part of U.S. Ser. No. 09/795,286, filed Oct. 13, 2000, which is a continuation-in-part of Australian Patent Application PR0745, filed Oct. 13, 2000; U.S. Ser. No. 09/755,983 is also a continuation-in-part of U.S. Ser. No. 09/795,302, filed Oct. 13, 2000, which is a continuation-in-part application of PCT/AU00/00329, filed Apr. 17, 2000, which is an international filing of Australian patent application PP9778, filed Apr. 15, 1999, each of which is incorporated herein by reference.

### FIELD OF THE INVENTION

The present disclosure is in the field of production of autoimmune diseases and allergies in mammals. More particularly, the present disclosure is in the field of treating and eliminating autoimmune diseases and allergies through stimulation of the thymus.

### BACKGROUND

### The Immune System

The major function of the immune system is to distinguish “foreign” antigens from “self” and respond accordingly to protect the body against infection. This definition may also be stated as distinguishing “bad” molecules from “good.” In normal immune responses, the sequence of events involves dedicated antigen presenting cells (APC) capturing foreign antigen and processing it into small peptide fragments which are then presented in clefts of major histocompatibility complex (MHC) molecules on the APC surface. The MHC molecules can either be of class I expressed on all nucleated cells (recognized by cytotoxic T lymphocytes (Tc)) or of class II expressed primarily by cells of the immune system (recognized by helper T lymphocytes (Th)). Th cells recognize the MHC II/peptide complexes on APC and respond.

There are two types of Th cells distinguished by the different types of soluble regulatory factors they produce. Th1 cells primarily produce IL2 and gamma interferon (IFNK). The latter, if present at the time of initial contact of naïve T cells with antigen, promotes the preferential activation of cell-mediated immunity (primarily Tc). Th2 cells produce Interleukin 4 (IL4), Interleukin 5 (IL5) and Interleukin 10 (IL10) which induce humoral immunity via antibody producing B cells which are specific for the particular antigen. In some cases this can lead to inappropriate allergic responses through IgE production.

The importance of Th cells in virtually all immune responses is best illustrated in HIV/AIDS where their absence through destruction by the virus causes severe immune deficiency eventually leading to death. Given their central importance as immunoregulatory cells, the balance between Th1 and Th 2 cells can have profound impact on the nature of the immune response. Such imbalances can occur through developmental abnormalities or inappropriate activation at the onset of immune responses. This can lead to a variety of diseases such as allergies, cancer and autoimmunity.

The ability to recognize antigen is encompassed in a plasma membrane receptor in T and B lymphocytes. These receptors are generated randomly by a complex series of rearrangements of many possible genes, such that each individual T or B cell has a unique antigen receptor. This enormous potential diversity means that for any single antigen the body might encounter, multiple lymphocytes will be able to recognize it with varying degrees of binding strength (affinity) and respond to varying degrees. Since the antigen receptor specificity arises by chance, the problem thus arises as to why the body doesn't “self destruct” through lymphocytes reacting against self antigens. Fortunately there are several mechanisms which prevent the T and B cells from doing so—collectively they create a situation where the immune system is tolerant to self.

The most efficient form of self tolerance is to physically remove (kill) any potentially reactive lymphocytes at the sites where they are produced (thymus for T cells, bone marrow for B cells). This is called central tolerance. An important, additional method of tolerance is through regulatory Th cells which inhibit autoreactive cells either directly, or more likely, through cytokines. Given that virtually all immune responses require initiation and regulation by T helper cells, a major aim of any tolerance induction regime would be to target these cells. Similarly, since Tc's are very important effector cells, their production is a major aim of strategies for e.g. anti-cancer and anti-viral therapy.

### The Thymus

The thymus is arguably the major organ in the immune system because it is the primary site of production of T lymphocytes. Its role is to attract appropriate bone marrow-derived precursor cells from the blood, and induce their commitment to the T cell lineage including the gene rearrangements necessary for the production of the T cell receptor for antigen (TCR). Associated with this is a remarkable degree of cell division to expand the number of T cells and hence increase the likelihood that every foreign antigen will be recognized and eliminated. A strange feature of T cell recognition of antigen, however, is that unlike B cells, the TCR only recognizes peptide fragments physically associated with MHC molecules; normally this is self MHC and this ability is selected for in the thymus. This process is called positive selection and is an exclusive feature of cortical epithelial cells. If the TCR fails to bind to the self MHC/peptide complexes, the T cell dies by “neglect.” It needs some degree of signaling through the TCR for its continued maturation.

While the thymus is fundamental for a functional immune system, releasing˜1% of its T cell content into the bloodstream per day, one of the apparent anomalies of mammals is that this organ undergoes severe atrophy as a result of sex steroid production. This can begin even in young children but is profound from the time of puberty. For normal healthy individuals this loss of production and release of new T cells does not always have clinical consequences (although immune-based disorders increase in incidence and severity with age). When there is a major loss of T cells, e.g., in AIDS and following chemotherapy or radiotherapy, the patients are highly susceptible to disease because they are immune suppressed.

Many T cells will develop, however, which by chance can recognize, with high affinity, self MHC/peptide complexes. Such T cells are thus potentially self-reactive and could cause severe autoimmune diseases such as multiple sclerosis, arthritis, diabetes, thyroiditis and systemic lupus erythematosis (SLE). Fortunately, if the affinity in the thymus of the TCR to self MHC/peptide complexes is too high, the developing thymocyte is induced to undergo a suicidal activation and dies by apoptosis, a process called negative selection. This is called central tolerance. Such T cells die rather than respond because in the thymus they are still immature. The most potent inducers of this negative selection in the thymus are APC called dendritic cells (DC). Being APC they deliver the strongest signal to the T cells; in the thymus this causes deletion, in the peripheral lymphoid organs where the T cells are more mature, the DC cause activation.

An important, additional method of tolerance is through the regulatory Th cells which can inhibit autoreactive cells through cytokines. Given that virtually all immune responses require initiation and regulation by T helper cells, a major aim of any therapeutic regime for treatment of many immune based disorders would be to target these cells. Similarly, since Tc are very important effector cells, their production is a major aim of strategies for, e.g., anti-cancer and anti-viral therapy. Depending on the nature of the Th defect, the resulting immune response could be excessive due to hyperreactive T and B cells or too weak due to hyporeactive T cells.

Very important clinical conditions which have hyperreactive T cells as their basis are the self destruction diseases (autoimmunity), hypersensitivities and allergies including asthma, dermatitis and psoriasis. Precisely how such diseases begin is unclear, but autoimmunity can at least in part result from infections, with the stimulating foreign antigen mimicking a self molecule. The immune cells then destroy both the foreign pathogen but also normal tissues. After the infectious reagent has been removed, the autoreactive T cells continue to destroy the self tissue. Allergies and hypersensitivities can result from inappropriate activation of Th1 or Th2, the associated cytokines causing pathological responses rather than beneficial responses. Such diseases can be treated by depleting all the T lymphocytes (which would include those causing the disease). The problem, however, is that the patients are then immune suppressed because of the lack of T cells. Furthermore they are incapable of producing significant numbers of new T cells because their thymus has severely atrophied from sex steroids. Attempts have been made to rescue such patients with hematopoietic stem cells but these have met with only limited success because of the lack of thymic function.

### Thymus Atrophy

The thymus is influenced to a great extent by its bidirectional communication with the neuroendocrine system (Kendall, 1988). Of particular importance is the interplay between the pituitary, adrenals and gonads on thymic function including both trophic (thyroid stimulating hormone or TSH and growth hormone or GH) and atrophic effects (leutinizing hormone or LH, follicle stimulating hormone or FSH and adrenocorticotropic hormone or ACTH) (Kendall, 1988; Homo-Delarche, 1991). Indeed one of the characteristic features of thymic physiology is the progressive decline in structure and function which is commensurate with the increase in circulating sex steroid production around puberty (Hirokawa and Makinodan, 1975; Tosi et al., 1982 and Hirokawa, et al., 1994). The precise target of the hormones and the mechanism by which they induce thymus atrophy is yet to be determined. Since the thymus is the primary site for the production and maintenance of the peripheral T cell pool, this atrophy has been widely postulated as the primary cause of an increased incidence of immune-based disorders in the elderly. In particular, deficiencies of the immune system illustrated by a decrease in T-cell dependent immune functions such as cytolytic T-cell activity and mitogenic responses, are reflected by an increased incidence of immunodeficiency, autoimmunity and tumor load in later life (Hirokawa, 1998).

The impact of thymus atrophy is reflected in the periphery, with reduced thymic input to the T cell pool resulting in a less diverse T cell receptor (TCR) repertoire. Altered cytokine profile (Hobbs et al., 1993; Kurashima et al., 1995), changes in CD4⁺ and CD8⁺ subsets and a bias towards memory as opposed to naïve T cells (Mackall et al., 1995) are also observed. Furthermore, the efficiency of thymopoiesis is impaired with age such that the ability of the immune system to regenerate normal T-cell numbers after T-cell depletion is eventually lost (Mackall et al., 1995). However, recent work by Douek et al. (1998), has shown presumably thymic output to occur even in old age in humans. Excisional DNA products of TCR gene-rearrangement were used to demonstrate circulating, de novo produced naïve T cells after HIV infection in older patients. The rate of this output and subsequent peripheral T cell pool regeneration needs to be further addressed since patients who have undergone chemotherapy show a greatly reduced rate of regeneration of the T cell pool, particularly CD4⁺ T cells, in postpubertal patients compared to those who were pre-pubertal (Mackall et al, 1995). This is further exemplified in recent work by Timm and Thoman (1999), who have shown that although CD4⁺ T cells are regenerated in old mice post bone marrow transplant (BMT), they appear to show a bias towards memory cells due to the aged peripheral microenvironment, coupled to poor thymic production of naïve T cells.

The thymus essentially consists of developing thymocytes interspersed within the diverse stromal cells (predominantly epithelial cell subsets) which constitute the microenvironment and provide the growth factors and cellular interactions necessary for the optimal development of the T cells. The symbiotic developmental relationship between thymocytes and the epithelial subsets that controls their differentiation and maturation (Boyd et al., 1993), means sex-steroid inhibition could occur at the level of either cell type which would then influence the status of the other. It is less likely that there is an inherent defect within the thymocytes themselves since previous studies, utilizing radiation chimeras, have shown that bone marrow (BM) stem cells are not affected by age (Hirokawa, 1998; Mackall and Gress, 1997) and have a similar degree of thymus repopulation potential as young BM cells. Furthermore, thymocytes in older aged animals retain their ability to differentiate to at least some degree (Mackall and Gress, 1997; George and Ritter, 1996; Hirokawa et al., 1994). However, recent work by Aspinall (1997), has shown a defect within the precursor CD3-CD4-CD8- triple negative (TN) population occurring at the stage of TCRK chain gene-rearrangement.

### SUMMARY OF THE INVENTION

The present disclosure concerns methods for destroying a patient's T cells to reduce clinical disease, where the disease is related to the presence of an abnormal set of T cells. This step is followed by thymic reactivation via blockage of sex steroid mediated signaling to the thymus. The degree and kinetics of thymic regrowth can be enhanced by injection of CD34+ hematopoietic stem cells (ideally autologous). The patient having been depleted of T cells will no longer have the disease and in the presence of a rapidly reforming thymus will soon produce a new cohort of T cells in the blood and lymphoid organs thereby providing immune protection against pathogens.

These methods are based on disrupting sex steroid mediated signaling to the thymus in the subject. In one embodiment castration is used to disrupt the sex steroid mediated signaling. In a preferred embodiment, chemical castration is used. In another embodiment surgical castration is used. Castration reverses the state of the thymus to its pre-pubertal state, thereby reactivating it.

In a particular embodiment sex steroid mediated signaling to the thymus is blocked by the administration of agonists or antagonists of LHRH, anti-estrogen antibodies, anti-androgen antibodies, passive (antibody) or active (antigen) anti-LHRH vaccinations, or combinations thereof (“blockers”).

In a preferred embodiment, the blocker(s) is administered by a sustained peptide-release formulation. Examples of sustained peptide-release formulations are provided in WO98/08533, the entire contents of which are incorporated herein by reference.

In a preferred embodiment hematopoietic or lymphoid stem and/or progenitor cells from a donor (preferably matched) are transplanted into the recipient to increase the speed of regeneration of the thymus. In another embodiment these cells are transplanted from a healthy donor, without autoimmune disease or allergies, to replace aberrant stem and/or progenitor cells in the patient.

In one preferred embodiment a patient's autoimmune disease is eliminated at least in part by clearance of the patient's T cell population. Sex steroid mediated signaling to the thymus is disrupted. Upon regeneration of the thymus and repopulation of the peripheral blood with new T cells, the aberrant T cells that failed to recognize self remain eliminated from the T cell population.

In another preferred embodiment a patient's allergies are eliminated by the same disruption of sex steroid mediated signaling to the thymus, followed by regeneration of the thymus and repopulation of the peripheral blood stream with a “clean” population of T cells.

### DESCRIPTION OF THE FIGURES

FIG. 1A and B: Changes in thymocyte number pre- and post-castration. Thymus atrophy results in a significant decrease in thymocyte numbers with age. By 2 weeks post-castration, cell numbers have increased to young adult levels. By 3 weeks post-castration, numbers have significantly increased from the young adult and they are stabilized by 4 weeks post-castration. ***=Significantly different from young adult (2 month) thymus, p<0.001

FIG. 2A-C: (A) Spleen numbers remain constant with age and post-castration. The B:T cell ratio in the periphery also remains constant (B), however, the CD4:CD8 ratio decreases significantly (p<0.001) with age and is restored to normal young levels by 4 weeks post-castration.

FIG. 3: Fluorescence Activated Cell Sorter (FACS) profiles of CD4 vs. CD8 thymocyte populations with age and post-castration. Percentages for each quadrant are given above each plot. Subpopulations of thymocytes remain constant with age and there is a synchronous expansion of thymocytes following castration.

FIG. 4: Proliferation of thymocytes as detected by incorporation of a pulse of BrdU. Proportion of proliferating thymocytes remains constant with age and following castration.

FIG. 5A-D: Effects of age and castration on proliferation of thymocyte subsets. (A) Proportion of each subset that constitutes the total proliferating population—The proportion of CD8+ T cells within the proliferating population is significantly increased. (B) Percentage of each subpopulation that is proliferating—The TN and CD8 Subsets have significantly less proliferation at 2 years than at 2 months. At 2 weeks post-castration, the TN population has returned to normal young levels of proliferation while the CD8 population shows a significant increase in proliferation. The level is equivalent to the normal young by 4 weeks post-castration. (C) Overall TN proliferation remains constant with age and post-castration. However (D) the significant decrease in proliferation of the TN1 subpopulation with age is not returned to normal levels by 4 weeks post-castration. ***=Highly significant, p<0.001, **=significant, p<0.01

FIG. 6: Mice were injected intrathymically with FITC. The number of FITC+ cells in the periphery were calculated 24 hours later. Although the proportion of recent thymic migrants (RTE) remained consistently about 1% of thymus cell number age but was significantly reduced at 2 weeks post-castration, there was a significant (p<0.01) decrease in the RTE cell numbers with age. Following castration, these values were increasing although still significantly lower than young mice at 2 weeks post-castration. With age, a significant increase in the ratio of CD4+ to CD8+ RTE was seen and this was normalized by 1 week post-castration.

FIG. 7A-C: Changes in thymus (A), spleen (B) and lymph node (C) cell numbers following treatment with cyclophosphamide, a chemotherapy agent. Note the rapid expansion of the thymus in castrated animals when compared to the non-castrate (cyclophosphamide alone) group at 1 and 2 weeks post-treatment. In addition, spleen and lymph node numbers of the castrate group were well increased compared to the cyclophosphamide alone group. By 4 weeks, cell numbers are normalized. (n=3-4 per treatment group and time point).

FIG. 8A-C: Changes in thymus (A), spleen (B) and lymph node (C) cell numbers following irradiation (625 Rads) one week after surgical castration. Note the rapid expansion of the thymus in castrated animals when compared to the non-castrate (irradiation alone) group at 1 and 2 weeks post-treatment. (n=3-4 per treatment group and time point).

FIG. 9A-C: Changes in thymus (A), spleen (B) and lymph node (C) cell numbers following irradiation and castration on the same day. Note the rapid expansion of the thymus in castrated animals when compared to the non-castrate group at 2 weeks post-treatment. However, the difference observed is not as obvious as when mice were castrated 1 week prior to treatment (FIG. 7). (n=3-4 per treatment group and time point).

FIG. 10: Changes in thymus (A), spleen (B) and lymph node (C) cell numbers following treatment with cyclophosphamide, a chemotherapy agent, and surgical or chemical castration performed on the same day. Note the rapid expansion of the thymus in castrated animals when compared to the non-castrate (cyclophosphamide alone) group at 1 and 2 weeks post-treatment. In addition, spleen and lymph node numbers of the castrate group were well increased compared to the cyclophosphamide alone group. (n=3-4 per treatment group and time point). Chemical castration is comparable to surgical castration in regeneration of the immune system post-cyclophosphamide treatment.

FIG. 11: Lymph node cellularity following foot-pad immunization with Herpes Simplex Virus-1 (HSV-1). Note the increased cellularity in the aged post-castration as compared to the aged non-castrated group. Bottom graph illustrates the overall activated cell number as gated on CD25 vs. CD8 cells by FACS.

FIG. 12A-C: Vβ10 expression on CTL (cytotoxic T lymphocytes) in activated LN (lymph nodes) following HSV-1 inoculation. Note the diminution of a clonal response in aged mice and the reinstatement of the expected response post-castration.

FIG. 13A-C: Castration restores responsiveness to HSV-1 immunization. (a) Aged mice showed a significant reduction in total lymph node cellularity post-infection when compared to both the young and post-castrate mice. (b) Representative FACS profiles of activated (CD8⁺CD25⁺) cells in the LN of HSV-1 infected mice. No difference was seen in proportions of activated CTL with age or post-castration. (c) The decreased cellularity within the lymph nodes of aged mice was reflected by a significant decrease in activated CTL numbers. Castration of the aged mice restored the immune response to HSV-1 with CTL numbers equivalent to young mice. Results are expressed as mean±1 SD of 8-12 mice. ** p≦0.01 compared to young (2-month) mice;ˆ p≦0.01 compared to aged (non-cx) mice.

FIG. 14: Popliteal lymph nodes were removed from mice immunized with HSV-1 and cultured for 3 days. CTL assays were performed with non-immunized mice as control for background levels of lysis (as determined by ⁵¹Cr-release). Results are expressed as mean of 8 mice, in triplicate±1SD. Aged mice showed a significant (p≦0.01, *) reduction in CTL activity at an E:T ratio of both 10:1 and 3:1 indicating a reduction in the percentage of specific CTL present within the lymph nodes. Castration of aged mice restored the CTL response to young adult levels.

FIG. 15A and B: Analysis of CD4⁺ T cell help and Vβ TCR response to HSV-1 infection. Popliteal lymph nodes were removed on D5 post-HSV-1 infection and analysed ex-vivo for the expression of (a) CD25, CD8 and specific TCRVβ markers and (b) CD4/CD8 T cells. (a) The percentage of activated (CD25⁺) CD8⁺ T cells expressing either Vβ10 or Vβ8.1 is shown as mean±1SD for 8 mice per group. No difference was observed with age or postcastration. (b) A decrease in CD4/CD8 ratio in the resting LN population was seen with age. This was restored post-castration. Results are expressed as mean±1SD of 8 mice per group. ***=p≦0.001 compared to young and castrate mice.

FIG. 16A-D: Changes in thymus (A), spleen (B), lymph node (C) and bone marrow (D) cell numbers following bone marrow transplantation of Ly5 congenic mice. Note the rapid expansion of the thymus in castrated animals when compared to the non-castrate group at all time points post-treatment. In addition, spleen and lymph node numbers of the castrate group were well increased compared to the cyclophosphamide alone group. (n=3-4 per treatment group and time point). Castrated mice had significantly increased congenic (Ly5.2) cells compared to non-castrated animals (data not shown).

FIG. 17A and B: Changes in thymus cell number in castrated and noncastrated mice after fetal liver reconstitution. (n=3-4 for each test group.) (A) At two weeks, thymus cell number of castrated mice was at normal levels and significantly higher than that of noncastrated mice (*p≦0.05). Hypertrophy was observed in thymuses of castrated mice after four weeks. Noncastrated cell numbers remain below control levels. (B) CD45.2⁺ cells-CD45.2⁺ is a marker showing donor derivation. Two weeks after reconstitution donor-derived cells were present in both castrated and noncastrated mice. Four weeks after treatment approximately 85% of cells in the castrated thymus were donor-derived. There were no donor-derived cells in the noncastrated thymus.

FIG. 18: FACS profiles of CD4 versus CD8 donor derived thymocyte populations after lethal irradiation and fetal liver reconstitution, followed by surgical castration. Percentages for each quadrant are given to the right of each plot. The age matched control profile is of an eight month old Ly5.1 congenic mouse thymus. Those of castrated and noncastrated mice are gated on CD45.2⁺ cells, showing only donor derived cells. Two weeks after reconstitution subpopulations of thymocytes do not differ between castrated and noncastrated mice.

FIG. 19A and B: Myeloid and lymphoid dendritic cell (DC) number after lethal irradiation, fetal liver reconstitution and castration. (n=3-4 mice for each test group.) Control (striped) bars on the following graphs are based on the normal number of dendritic cells found in untreated age matched mice. (A) Donor-derived myeloid dendritic cells—Two weeks after reconstitution DC were present at normal levels in noncastrated mice. There were significantly more DC in castrated mice at the same time point. (*p≦0.05). At four weeks DC number remained above control levels in castrated mice. (B) Donor-derived lymphoid dendritic cells—Two weeks after reconstitution DC numbers in castrated mice were double those of noncastrated mice. Four weeks after treatment DC numbers remained above control levels.

FIG. 20A and B: Changes in total and CD45.2⁺ bone marrow cell numbers in castrated and noncastrated mice after fetal liver reconstitution. n=3-4 mice for each test group. (A) Total cell number—Two weeks after reconstitution bone marrow cell numbers had normalized and there was no significant difference in cell number between castrated and noncastrated mice. Four weeks after reconstitution there was a significant difference in cell number between castrated and noncastrated mice (*p≦0.05). (B) CD45.2⁺ cell number. There was no significant difference between castrated and noncastrated mice with respect to CD45.2+ cell number in the bone marrow two weeks after reconstitution. CD45.2⁺ cell number remained high in castrated mice at four weeks. There were no donor-derived cells in the noncastrated mice at the same time point.

FIG. 21A-C: Changes in T cells and myeloid and lymphoid derived dendritic cells (DC) in bone marrow of castrated and noncastrated mice after fetal liver reconstitution. (n−3-4 mice for each test group.) Control (striped) bars on the following graphs are based on the normal number of T cells and dendritic cells found in untreated age matched mice. (A) T cell number—Numbers were reduced two and four weeks after reconstitution in both castrated and noncastrated mice. (B) Donor derived myeloid dendritic cells—Two weeks after reconstitution DC cell numbers were normal in both castrated and noncastrated mice. At this time point there was no significant difference between numbers in castrated and noncastrated mice. (C) Donor-derived lymphoid dendritic cells—Numbers were at normal levels two and four weeks after reconstitution. At two weeks there was no significant difference between numbers in castrated and noncastrated mice.

FIG. 22A and B: Change in total and donor (CD45.2⁺) spleen cell numbers in castrated and noncastrated mice after fetal liver reconstitution. (n=3-4 mice for each test group.) (A) Total cell number—Two weeks after reconstitution cell numbers were decreased and there was no significant difference in cell number between castrated and noncastrated mice. Four weeks after reconstitution cell numbers were approaching normal levels in castrated mice. (B) CD45.2⁺ cell number—There was no significant difference between castrated and noncastrated mice with respect to CD45.2⁺ cell number in the spleen, two weeks after reconstitution. CD45.2⁺ cell number remained high in castrated mice at four weeks. There were no donor-derived cells in the noncastrated mice at the same time point.

FIG. 23A-C: Splenic T cells and myeloid and lymphoid derived dendritic cells (DC) after fetal liver reconstitution. (n=3-4 mice for each test group.) Control (striped) bars on the following graphs are based on the normal number of T cells and dendritic cells found in untreated age matched mice. (A) T cell number-Numbers were reduced two and four weeks after reconstitution in both castrated and noncastrated mice. (B) Donor derived (CD45.2⁺) myeloid dendritic cells—two and four weeks after reconstitution DC numbers were normal in both castrated and noncastrated mice. At two weeks there was no significant difference between numbers in castrated and noncastrated mice. (C) Donor-derived (CD45.2⁺) lymphoid dendritic cells—numbers were at normal levels two and four weeks after reconstitution. At two weeks there was no significant difference between numbers in castrated and noncastrated mice.

FIG. 24A and B: Changes in total and donor (CD45.2⁺) lymph node cell numbers in castrated and noncastrated mice after fetal liver reconstitution. (n=3-4 for each test group.) (A) Total cell numbers—Two weeks after reconstitution cell numbers were at normal levels and there was no significant difference between castrated and noncastrated mice. Four weeks after reconstitution cell numbers in castrated mice were at normal levels. (B) CD45.2⁺ cell number—There was no significant difference between castrated and noncastrated mice with respect to donor CD45.2⁺ cell number in the lymph node two weeks after reconstitution. CD45.2 cell number remained high in castrated mice at four weeks. There were no donor-derived cells in the noncastrated mice at the same point.

FIG. 25A-C: Changes in T cells and myeloid and lymphoid derived dendritic cells (DC) in the mesenteric lymph nodes of castrated and non-castrated mice after fetal liver reconstitution. (n=3-4 mice for each test group.) Control (striped) bars are the number of T cells and dendritic cells found in untreated age matched mice. (A) T cell numbers were reduced two and four weeks after reconstitution in both castrated and noncastrated mice. (B) Donor derived myeloid dendritic cells were normal in both castrated and noncastrated mice. At four weeks they were decreased. At two weeks there was no significant difference between numbers in castrated and noncastrated mice. (C) Donor-derived lymphoid dendritic cells—Numbers were at normal levels two and four weeks after reconstitution. At two weeks there was no significant difference between numbers in castrated and noncastrated mice.

FIG. 26: The phenotypic composition of peripheral blood lymphocytes was analyzed in human patients (all>60 years) undergoing LHRH agonist treatment for prostate cancer. Patient samples were analyzed before treatment and 4 months after beginning LHRH agonist treatment. Total lymphocyte cell numbers per ml of blood were at the lower end of control values before treatment in all patients. Following treatment, 6/9 patients showed substantial increases in total lymphocyte counts (in some cases a doubling of total cells was observed). Correlating with this was an increase in total T cell numbers in 6/9 patients. Within the CD4⁺ subset, this increase was even more pronounced with 8/9 patients demonstrating increased levels of CD4 T cells. A less distinctive trend was seen within the CD8⁺ subset with 4/9 patients showing increased levels, albeit generally to a smaller extent than CD4⁺ T cells.

FIG. 27: Analysis of human patient blood before and after LHRH-agonist treatment demonstrated no substantial changes in the overall proportion of T cells, CD4 or CD8 T cells, and a variable change in the CD4:CD8 ratio following treatment. This indicates the minimal effect of treatment on the homeostatic maintenance of T cell subsets despite the substantial increase in overall T cell numbers following treatment. All values were comparative to control values.

FIG. 28: Analysis of the proportions of B cells and myeloid cells (NK, NKT and macrophages) within the peripheral blood of human patients undergoing LHRH agonist treatment demonstrated a varying degree of change within subsets. While NK, NKT and macrophage proportions remained relatively constant following treatment, the proportion of B cells was decreased in 4/9 patients.

FIG. 29: Analysis of the total cell numbers of B and myeloid cells within the peripheral blood of human patients post-treatment showed clearly increased levels of NK (5/9 patients), NKT (4/9 patients) and macrophage (3/9 patients) cell numbers post-treatment. B cell numbers showed no distinct trend with 2/9 patients showing increased levels; 4/9 patients showing no change and 3/9 patients showing decreased levels.

FIG. 30A and B: The major change seen post-LHRH agonist treatment was within the T cell population of the peripheral blood. In particular there was a selective increase in the proportion of naïve (CD45RA⁺) CD4+ cells, with the ratio of naïve (CD45RA⁺) to memory (CD45RO⁺) in the CD4⁺ T cell subset increasing in 6/9 of the human patients.

FIG. 31: Decrease in the impedance of skin using various laser pulse energies. There is a decrease in skin impedance in skin irradiated at energies as low as 10 mJ, using the fitted curve to interpolate data.

FIG. 32: Permeation of a pharmaceutical through skin. Permeability of the skin, using insulin as a sample pharmaceutical, was greatly increased through laser irradiation.

FIG. 33: Change in fluorescence of skin over time after the addition of 5-aminolevulenic acid (ALA) and a single impulse transient to the skin. The peak of intensity occurs at about 640 nm and is highest after 210 minutes (dashed line) post-treatment.

FIG. 34: Change in fluorescence of skin over time after the addition of 5-aminolevulenic acid (ALA) without an impulse transient. There is little change in the intensity at different time points.

FIG. 35: Comparison of change in fluorescence of skin after the addition of 5-aminolevulenic acid (ALA) and a single impulse transient under various peak stresses. The degree of permeabilization of the stratum corneum depends on the peak stress.

### DETAILED DESCRIPTION OF THE INVENTION

The present disclosure provides methods for treating and eliminating autoimmune diseases and allergies. This is based on the fact that both of these types of diseases are caused by aberrance within a patient's T cell population. By destroying the patient's current compliment of T cells followed by reactivating the patient's thymus using the methods of this invention, the aberrant T cells are removed and replaced with a normal set of T cells. The patient's thymus is reactivated by disruption of sex steroid mediated signaling to the thymus. This disruption reverses the hormonal status of the patient. A preferred method for creating disruption is through castration. During or after the castration step, hematopoietic stem or progenitor cells may be transplanted into the patient. Methods of castration include but are not limited to chemical castration and surgical castration. Upon reactivation of the thymus a new set of T cells, without the aberrant group, are created.

A preferred method of reactivating the thymus is by blocking the direct and/or indirect stimulatory effects of LHRH on the pituitary, which leads to a loss of the gonadotrophins FSH and LH. These gonadotrophins normally act on the gonads to release sex hormones, in particular estrogens in females and testosterone in males; the release is blocked by the loss of FSH and LH. The direct consequences of this are an immediate drop in the plasma levels of sex steroids, and as a result, progressive release of the inhibitory signals on the thymus. The degree and kinetics of thymic regrowth can be enhanced by injection of CD34⁺ hematopoietic cells (ideally autologous).

This invention may be used with any animal species (including humans) having sex steroid driven maturation and an immune system, such as mammals and marsupials, preferably large mammals, and most preferably humans.

The terms “regeneration,” “reactivation” and “reconstitution” and their derivatives are used interchangeably herein, and refer to the recovery of an atrophied thymus to its active state.

The term “donor” refers to the source of cells that may be provided to the patient. The donor may be syngeneic, allogeneic or xenogeneic. Allogeneic donors are preferred. Allogeneic donors are unmatched members of the same species, while xenogeneic donors are of a different species from the patient. Syngeneic donors, matched with the patient, are the most preferred. The terms “matched,” “unmatched, “mismatched,” and non-identical” with reference to donors or donor cells are used to indicate that the MHC and/or minor histocompatibility markers of the donor and the patient are (matched) or are not (unmatched, mismatched and non-identical) the same.

“Castration,” as used herein, means the marked reduction or elimination of sex steroid production and distribution in the body. This effectively returns the patient to prepubertal status when the thymus is fully functioning. Surgical castration removes the patient's gonads.

A less permanent version of castration is through the administration of a chemical for a period of time, referred to herein as “chemical castration.” A variety of chemicals are capable of functioning in this manner. During the chemical delivery, and for a period of time afterwards, the patient's hormone production is turned off. Preferably the castration is reversed upon termination of chemical delivery.

As detailed below, the present disclosure shows that the sex steroid-induced atrophic thymus can be dramatically restored structurally and functionally to approximately its optimal capacity. The newly generated T cells emigrate from the thymus and restore peripheral T cell levels and function in the periphery. A major feature of the increased thymic activity is the involvement of appropriate blood-derived hematopoietic precursor cells which enter the thymus and give rise to all the thymocyte subsets. These precursors can either be host or donor-derived; the latter do not have to be identical to the host. This means that any genetic defect in the host hematopoietic cells can be overcome by transfer of donor stem cells.

In particular, the following procedure can be used. The patient with inappropriate T cell function (in most cases this will be hyperreactivity causing diseases such as allergies and autoimmunity) will undergo standard T cell depletion therapy based normally on the injection of anti-T cell antibodies. Because in many cases it is not possible to reduce only the antigen specific T cells which cause the disease, this approach is aimed at depleting initially all T cells (including the pathological ones). Simultaneously, however, the lack of T cells induces a state of generalized immunodeficiency which means the patients are highly susceptible to infection, particularly viral. Even B cell responses will not function normally in the absence of appropriate T cell help. To overcome this, the patient will be given sex steroid ablation therapy, commonly in the form of LHRH agonist or antagonist. Reduction of sex steroids in the blood to minimal values is expected to take 2-3 weeks; concordant with this is the reactivation of the thymus.

Blood precursor cells (ideally as CD34+ sorted population) can be injected into the patients at around this time or slightly earlier. These cells can be derived from a syngeneic, allogeneic or xenogeneic donor. The levels of these CD34+ cells in the donor blood can be increased by prior injection of the donor and/or the patient with G-CSF. They can also be obtained by sorting from donor bone marrow.

The reactivated thymus will take up the precursor cells and convert them into new T cells which will emigrate into the blood stream and re-build a normal immune system in the patient. If the patient is genetically prone to disease, this may be overcome by using hematopoietic stem cells (HSC) from a healthy donor. Because the HSC have a strong self-renewal capacity, the hematopoietic chimera so formed will be stable theoretically for the life of the patient (as for normal people).

It is expected that within 3-4 weeks of LHRH therapy the first new T cells will be present in the blood stream but the therapy will be maintained for 3-4 months to allow complete normalization of the immune system.

This therapy can be combined with other approaches to blocking or modifying T cell activation and introduction of cytokines to modify the regenerating immune system.

### Disruption of Sex Steroid Mediated Signaling to the Thymus

As will be readily understood, sex steroid mediated signaling to the thymus can be disrupted in a range of ways well known to those of skill in the art, some of which are described herein. For example, inhibition of sex steroid production or blocking of one or more sex steroid receptors within the thymus will accomplish the desired disruption, as will administration of sex steroid agonists or antagonists, or active (antigen) or passive (antibody) anti-sex steroid vaccinations. Inhibition of sex steroid production can also be achieved by administration of one or more sex steroid analogs. In some clinical cases, permanent removal of the gonads via physical castration may be appropriate.

In a preferred embodiment, the sex steroid mediated signaling to the thymus is disrupted by administration of a sex steroid analog, preferably an analog of luteinizing hormone-releasing hormone (LHRH). Sex steroid analogs and their use in therapies and chemical castration are well known. Such analogs include, but are not limited to, the following agonists of the LHRH receptor (LHRH-R): Buserelin (Hoechst), Cystorelin (Hoechst), Decapeptyl (trade name Debiopharm; Ipsen/Beaufour), Deslorelin (Balance Pharmaceuticals), Gonadorelin (Ayerst), Goserelin (trade name Zoladex; Zeneca), Histrelin (Ortho), Leuprolide (trade name Lupron; Abbott/TAP), Leuprorelin (Plosker et al.), Lutrelin (Wyeth), Meterelin (WO9118016), Nafarelin (Syntex), and Triptorelin (U.S. Pat. No. 4,010,125). LHRH analogs also include, but are not limited to, the following antagonists of the LHRH-R: Abarelix (trade name Plenaxis; Praecis) and Cetrorelix (trade name; Zentaris). Combinations of agonists, combinations of antagonists, and combinations of agonists and antagonists are also included. The disclosures of each the references referred to above are incorporated herein by reference. It is currently preferred that the analog is Deslorelin (described in U.S. Pat. No. 4,218,439). For a more extensive list, see Vickery et al., 1984.

In a preferred embodiment, an LHRH receptor (LHRH-R) antagonist is delivered to the patient, followed by an LHRH-R agonist. This protocol will abolish or limit any spike of sex steroid production, before the decrease in sex steroid production, that might be produced by the administration of the agonist. In an alternate embodiment, an LHRH-R agonist that creates little or no sex steroid production spike is used, with or without the prior administration of an LHRH-R antagonist.

While the stimulus for thymic reactivation is fundamentally based on the inhibition of the effects of sex steroids and/or the direct effects of the LHRH analogs, it may be useful to include additional substances which can act in concert to enhance the thymic effect. Such compounds include but are not limited to Interleukin 2 (IL2), Interleukin 7 (IL7), Interleukin 15 (IL15), members of the epithelial and fibroblast growth factor familes, Stem Cell Factor, granulocyte colony stimulating factor (GCSF) and keratinocyte growth factor (KGF). It is envisaged that these additional compound(s) would only be given once at the initial LHRH analog application. However, additional doses of any one or combination of these substances may be given at any time to further stimulate the thymus. In addition, steroid receptor based modulators, which may be targeted to be thymic specific, may be developed and used.

### Pharmaceutical Compositions

The compounds used in this invention can be supplied in any pharmaceutically acceptable carrier or without a carrier. Examples include physiologically compatible coatings, solvents and diluents. For parenteral, subcutaneous, intravenous and intramuscular administration, the compositions may be protected such as by encapsulation. Alternatively, the compositions may be provided with carriers that protect the active ingredient(s), while allowing a slow release of those ingredients. Numerous polymers and copolymers are known in the art for preparing time-release preparations, such as various versions of lactic acid/glycolic acid copolymers. See, for example, U.S. Pat. No. 5,410,016, which uses modified polymers of polyethylene glycol (PEG) as a biodegradeable coating.

Formulations intended to be delivered orally can be prepared as liquids, capsules, tablets, and the like. These compositions can include, for example, excipients, diluents, and/or coverings that protect the active ingredient(s) from decomposition. Such formulations are well known.

In any of the formulations, other compounds that do not negatively affect the activity of the LHRH analogs may be included. Examples are various growth factors and other cytokines as described herein.

### Dose

The LHRH analog can be administered in a one-time dose that will last for a period of time. Preferably, the formulation will be effective for one to two months. The standard dose varies with type of analog used. In general, the dose is between about 0.01 μg/kg and about 10 mg/kg, preferably between about 0.01 mg/kg and about 5 mg/kg. Dose varies with the LHRH analog or vaccine used. In a preferred embodiment, a dose is prepared to last as long as a periodic epidemic lasts. For example, “flu season” occurs usually during the winter months. A formulation of an LHRH analog can be made and delivered as described herein to protect a patient for a period of two or more months starting at the beginning of the flu season, with additional doses delivered every two or more months until the risk of infection decreases or disappears.

The formulation can be made to enhance the immune system. Alternatively, the formulation can be prepared to specifically deter infection by flu viruses while enhancing the immune system. This latter formulation would include GM cells that have been engineered to create resistance to flu viruses (see below). The GM cells can be administered with the LHRH analog formulation or separately, both spatially and/or in time. As with the non-GM cells, multiple doses over time can be administered to a patient to create protection and prevent infection with the flu virus over the length of the flu season.

The reactivation of the thymus can be supplemented by the addition of CD34⁺ hematopoietic stem cells (HSC) and/or epithelial stem cells (ESC) slightly before or at the time the thymus begins to regenerate. Ideally these cells are autologous or syngeneic and have been obtained from the patient or twin prior to thymus reactivation. The HSC can be obtained by sorting CD34⁺ cells from the patient's blood and/or bone marrow. The number of HSC can be enhanced in several ways, including (but not limited to) by administering G-CSF (Neupogen, Amgen) to the patient prior to collecting cells, culturing the collected cells in Stem Cell Growth Factor, and/or administering G-CSF to the patient after CD34⁺ cell supplementation. Alternatively, the CD34⁺ cells need not be sorted from the blood or BM if their population is enhanced by prior injection of G-CSF into the patient.

### Delivery of Agents for Chemical Castration

Delivery of the compounds of this invention can be accomplished via a number of methods known to persons skilled in the art. One standard procedure for administering chemical inhibitors to inhibit sex steroid mediated signaling to the thymus utilizes a single dose of an LHRH agonist that is effective for three months. For this a simple one-time i.v. or i.m. injection would not be sufficient as the agonist would be cleared from the patient's body well before the three months are over. Instead, a depot injection or an implant may be used, or any other means of delivery of the inhibitor that will allow slow release of the inhibitor. Likewise, a method for increasing the half life of the inhibitor within the body, such as by modification of the chemical, while retaining the function required herein, may be used.

Examples of more useful delivery mechanisms include, but are not limited to, laser irradiation of the skin, and creation of high pressure impulse transients (also called stress waves or impulse transients) on the skin, each method accompanied or followed by placement of the compound(s) with or without carrier at the same locus. A preferred method of this placement is in a patch placed and maintained on the skin for the duration of the treatment.

One means of delivery utilizes a laser beam, specifically focused, and lasing at an appropriate wavelength, to create small perforations or alterations in the skin of a patient. See U.S. Pat. No. 4,775,361, U.S. Pat. No. 5,643,252, U.S. Pat. No. 5,839,446, and U.S. Pat. No. 6,056,738, all of which are incorporated herein by reference. In a preferred embodiment, the laser beam has a wavelength between 0.2 and 10 microns. More preferably, the wavelength is between about 1.5 and 3.0 microns. Most preferably the wavelength is about 2.94 microns. In one embodiment, the laser beam is focused with a lens to produce an irradiation spot on the skin through the epidermis of the skin. In an additional embodiment, the laser beam is focused to create an irradiation spot only through the stratum corneum of the skin.

As used herein, “ablation” and “perforation” mean a hole created in the skin. Such a hole can vary in depth; for example it may only penetrate the stratum corneum, it may penetrate all the way into the capillary layer of the skin, or it may terminate anywhere in between. As used herein, “alteration” means a change in the skin structure, without the creation of a hole, that increases the permeability of the skin. As with perforation, skin can be altered to any depth.

Several factors may be considered in defining the laser beam, including wavelength, energy fluence, pulse temporal width and irradiation spot-size. In a preferred embodiment, the energy fluence is in the range of 0.03-100,000 J/cm². More preferably, the energy fluence is in the range of 0.03-9.6 J/cm². The beam wavelength is dependent in part on the laser material, such as Er:YAG. The pulse temporal width is a consequence of the pulse width produced by, for example, a bank of capacitors, the flashlamp, and the laser rod material. The pulse width is optimally between 1 fs (femtosecond) and 1,000 μs.

According to this method the perforation or alteration produced by the laser need not be produced with a single pulse from the laser. In a preferred embodiment a perforation or alteration through the stratum corneum is produced by using multiple laser pulses, each of which perforates or alters only a fraction of the target tissue thickness.

To this end, one can roughly estimate the energy required to perforate or alter the stratum corneum with multiple pulses by taking the energy in a single pulse and dividing by the number of pulses desirable. For example, if a spot of a particular size requires 1 J of energy to produce a perforation or alteration through the entire stratum corneum, then one can produce qualitatively similar perforation or alteration using ten pulses, each having 1/10th the energy. Because it is desirable that the patient not move the target tissue during the irradiation (human reaction times are on the order of 100 ms or so), and that the heat produced during each pulse not significantly diffuse, in a preferred embodiment the pulse repetition rate from the laser should be such that complete perforation is produced in a time of less than 100 ms. Alternatively, the orientation of the target tissue and the laser can be mechanically fixed so that changes in the target location do not occur during the longer irradiation time.

To penetrate the skin in a manner that induces little or no blood flow, skin can be perforated or altered through the outer surface, such as the stratum corneum layer, but not as deep as the capillary layer. The laser beam is focussed precisely on the skin, creating a beam diameter at the skin in the range of approximately 0.5 microns-5.0 cm. Optionally, the spot can be slit-shaped, with a width of about 0.05-0.5 mm and a length of up to 2.5 mm. The width can be of any size, being controlled by the anatomy of the area irradiated and the desired permeation rate of the fluid to be removed or the pharmaceutical to be applied. The focal length of the focusing lens can be of any length, but in one embodiment it is 30 mm.

By modifying wavelength, pulse length, energy fluence (which is a function of the laser energy output (in Joules) and size of the beam at the focal point (cm²)), and irradiation spot size, it is possible to vary the effect on the stratum corneum between ablation (perforation) and non-ablative modification (alteration). Both ablation and non-ablative alteration of the stratum corneum result in enhanced permeation of subsequently applied pharmaceuticals.

For example, by reducing the pulse energy while holding other variables constant, it is possible to change between ablative and non-ablative tissue-effect. Using an Er:YAG laser having a pulse length of about 300 μs, with a single pulse or radiant energy and irradiating a 2 mm spot on the skin, a pulse energy above approximately 100 mJ causes partial or complete ablation, while any pulse energy below approximately 100 mJ causes partial ablation or non-ablative alteration to the stratum corneum. Optionally, by using multiple pulses, the threshold pulse energy required to enhance permeation of body fluids or for pharmaceutical delivery is reduced by a factor approximately equal to the number of pulses.

Alternatively, by reducing the spot size while holding other variables constant, it is also possible to change between ablative and non-ablative tissue-effect. For example, halving the spot area will result in halving the energy required to produce the same effect. Irradiation down to 0.5 microns can be obtained, for example, by coupling the radiant output of the laser into the objective lens of a microscope objective. (e.g., as available from Nikon, Inc., Melville, N.Y.). In such a case, it is possible to focus the beam down to spots on the order of the limit of resolution of the microscope, which is perhaps on the order of about 0.5 microns. In fact, if the beam profile is Gaussian, the size of the affected irradiated area can be less than the measured beam size and can exceed the imaging resolution of the microscope. To non-ablatively alter tissue in this case, it would be suitable to use a 3.2 J/cm² energy fluence, which for a half-micron spot size would require a pulse energy of about 5 nJ. This low a pulse energy is readily available from diode lasers, and can also be obtained from, for example, the Er:YAG laser by attenuating the beam by an absorbing filter, such as glass.

Optionally, by changing the wavelength of radiant energy while holding the other variables constant, it is possible to change between an ablative and non-ablative tissue-effect. For example, using Ho:YAG (holmium: YAG; 2.127 microns) in place of the Er:YAG (erbium: YAG; 2.94 microns) laser, would result in less absorption of energy by the tissue, creating less of a perforation or alteration.

Picosecond and femtosecond pulses produced by lasers can also be used to produce alteration or ablation in skin. This can be accomplished with modulated diode or related microchip lasers, which deliver single pulses with temporal widths in the 1 femtosecond to 1 ms range. (See D. Stem et al., “Corneal Ablation by Nanosecond, Picosecond, and Femtosecond Lasers at 532 and 625 nm,” Corneal Laser Ablation, Vol. 107, pp. 587-592 (1989), incorporated herein by reference, which discloses the use of pulse lengths down to 1 femtosecond).

Another delivery method uses high pressure impulse transients on skin to create permeability. See U.S. Pat. No. 5,614,502, and U.S. Pat. No. 5,658,892, both of which are incorporated herein by reference. High pressure impulse transients, e.g., stress waves (e.g., laser stress waves (LSW) when generated by a laser), with specific rise times and peak stresses (or pressures), can safely and efficiently effect the transport of compounds, such as those of the present disclosure, through layers of epithelial tissues, such as the stratum corneum and mucosal membranes. These methods can be used to deliver compounds of a wide range of sizes regardless of their net charge. In addition, impulse transients used in the present methods avoid tissue injury.

Prior to exposure to an impulse transient, an epithelial tissue layer, e.g., the stratum corneum, is likely impermeable to a foreign compound; this prevents diffusion of the compound into cells underlying the epithelial layer. Exposure of the epithelial layer to the impulse transients enables the compound to diffuse through the epithelial layer. The rate of diffusion, in general, is dictated by the nature of the impulse transients and the size of the compound to be delivered.

The rate of penetration through specific epithelial tissue layers, such as the stratum corneum of the skin, also depends on several other factors including pH, the metabolism of the cutaneous substrate tissue, pressure differences between the region external to the stratum corneum, and the region internal to the stratum corneum, as well as the anatomical site and physical condition of the skin. In turn, the physical condition of the skin depends on health, age, sex, race, skin care, and history. For example, prior contacts with organic solvents or surfactants affect the physical condition of the skin.

The amount of compound delivered through the epithelial tissue layer will also depend on the length of time the epithelial layer remains permeable, and the size of the surface area of the epithelial layer which is made permeable.

The properties and characteristics of impulse transients are controlled by the energy source used to create them. See WO 98/23325, which is incorporated herein by reference. However, their characteristics are modified by the linear and non-linear properties of the coupling medium through which they propagate. The linear attenuation caused by the coupling medium attenuates predominantly the high frequency components of the impulse transients. This causes the bandwidth to decrease with a corresponding increase in the rise time of the impulse transient. The non-linear properties of the coupling medium, on the other hand, cause the rise time to decrease. The decrease of the rise time is the result of the dependence of the sound and particle velocity on stress (pressure). As the stress increases, the sound and the particle velocity increase as well. This causes the leading edge of the impulse transient to become steeper. The relative strengths of the linear attenuation, non-linear coefficient, and the peak stress determine how long the wave has to travel for the increase in steepness of rise time to become substantial.

The rise time, magnitude, and duration of the impulse transient are chosen to create a non-destructive (i.e., non-shock wave) impulse transient that temporarily increases the permeability of the epithelial tissue layer. Generally the rise time is at least 1 ns, and is more preferably about 10 ns.

The peak stress or pressure of the impulse transients varies for different epithelial tissue or cell layers. For example, to transport compounds through the stratum corneum, the peak stress or pressure of the impulse transient should be set to at least 400 bar; more preferably at least 1,000 bar, but no more than about 2,000 bar. For epithelial mucosal layers, the peak pressure should be set to between 300 bar and 800 bar, and is preferably between 300 bar and 600 bar. The impulse transients preferably have durations on the order of a few tens of ns, and thus interact with the epithelial tissue for only a short period of time. Following interaction with the impulse transient, the epithelial tissue is not permanently damaged, but remains permeable for up to about three minutes.

In addition, these methods involve the application of only a few discrete high amplitude pulses to the patient. The number of impulse transients administered to the patient is typically less than 100, more preferably less than 50, and most preferably less than 10. When multiple optical pulses are used to generate the impulse transient, the time duration between sequential pulses is 10 to 120 seconds, which is long enough to prevent permanent damage to the epithelial tissue.

Properties of impulse transients can be measured using methods standard in the art. For example, peak stress or pressure, and rise time can be measured using a polyvinylidene fluoride (PVDF) transducer method as described in Doukas et al., Ultrasound Med. Biol., 21:961 (1995).

Impulse transients can be generated by various energy sources. The physical phenomenon responsible for launching the impulse transient is, in general, chosen from three different mechanisms: (1) thermoelastic generation; (2) optical breakdown; or (3) ablation.

For example, the impulse transients can be initiated by applying a high energy laser source to ablate a target material, and the impulse transient is then coupled to an epithelial tissue or cell layer by a coupling medium. The coupling medium can be, for example, a liquid or a gel, as long as it is non-linear. Thus, water, oil such as castor oil, an isotonic medium such as phosphate buffered saline (PBS), or a gel such as a collagenous gel, can be used as the coupling medium.

In addition, the coupling medium can include a surfactant that enhances transport, e.g., by prolonging the period of time in which the stratum corneum remains permeable to the compound following the generation of an impulse transient. The surfactant can be, e.g., ionic detergents or nonionic detergents and thus can include, e.g., sodium lauryl sulfate, cetyl trimethyl ammonium bromide, and lauryl dimethyl amine oxide.

The absorbing target material acts as an optically triggered transducer. Following absorption of light, the target material undergoes rapid thermal expansion, or is ablated, to launch an impulse transient. Typically, metal and polymer films have high absorption coefficients in the visible and ultraviolet spectral regions.

Many types of materials can be used as the target material in conjunction with a laser beam, provided they fully absorb light at the wavelength of the laser used. The target material can be composed of a metal such as aluminum or copper; a plastic, such as polystyrene, e.g., black polystyrene; a ceramic; or a highly concentrated dye solution. The target material must have dimensions larger than the cross-sectional area of the applied laser energy. In addition, the target material must be thicker than the optical penetration depth so that no light strikes the surface of the skin. The target material must also be sufficiently thick to provide mechanical support. When the target material is made of a metal, the typical thickness will be 1/32 to 1/16 inch. For plastic target materials, the thickness will be 1/16 to ⅛ inch.

Impulse transients can also be enhanced using confined ablation. In confined ablation, a laser beam transparent material, such as a quartz optical window, is placed in close contact with the target material. Confinement of the plasma, created by ablating the target material by using the transparent material, increases the coupling coefficient by an order of magnitude (Fabro et al., J. Appl. Phys., 68:775, 1990). The transparent material can be quartz, glass, or transparent plastic.

Since voids between the target material and the confining transparent material allow the plasma to expand, and thus decrease the momentum imparted to the target, the transparent material is preferably bonded to the target material using an initially liquid adhesive, such as carbon-containing epoxies, to prevent such voids.

The laser beam can be generated by standard optical modulation techniques known in the art, such as by employing Q-switched or mode-locked lasers using, for example, electro- or acousto-optic devices. Standard commercially available lasers that can operate in a pulsed mode in the infrared, visible, and/or infrared spectrum include Nd:YAG, Nd:YLF, C0₂, excimer, dye, Ti:sapphire, diode, holmium (and other rare-earth materials), and metal-vapor lasers. The pulse widths of these light sources are adjustable, and can vary from several tens of picoseconds (ps) to several hundred microseconds. For use in the present disclosure, the optical pulse width can vary from 100 ps to about 200 ns and is preferably between about 500 ps and 40 ns.

Impulse transients can also be generated by extracorporeal lithotripters (one example is described in Coleman et al., Ultrasound Med. Biol., 15:213-227, 1989). These impulse transients have rise times of 30 to 450 ns, which is longer than laser-generated impulse transients. To form an impulse transient of the appropriate rise time for the new methods using an extracorporeal lithotripter, the impulse transient is propagated in a non-linear coupling medium (e.g., water) for a distance determined by equation (1), above. For example, when using a lithotripter creating an impulse transient having a rise time of 100 ns and a peak pressure of 500 barr, the distance that the impulse transient should travel through the coupling medium before contacting an epithelial cell layer is approximately 5 mm.

An additional advantage of this approach for shaping impulse transients generated by lithotripters is that the tensile component of the wave will be broadened and attenuated as a result of propagating through the non-linear coupling medium. This propagation distance should be adjusted to produce an impulse transient having a tensile component that has a pressure of only about 5 to 10% of the peak pressure of the compressive component of the wave. Thus, the shaped impulse transient will not damage tissue.

The type of lithotripter used is not critical. Either an electrohydraulic, electromagnetic, or piezoelectric lithotripter can be used.

The impulse transients can also be generated using transducers, such as piezoelectric transducers. Preferably, the transducer is in direct contact with the coupling medium, and undergoes rapid displacement following application of an optical, thermal, or electric field to generate the impulse transient. For example, dielectric breakdown can be used, and is typically induced by a high-voltage spark or piezoelectric transducer (similar to those used in certain extracorporeal lithotripters, Coleman et al., Ultrasound Med. Biol., 15:213-227, 1989). In the case of a piezoelectric transducer, the transducer undergoes rapid expansion following application of an electrical field to cause a rapid displacement in the coupling medium.

In addition, impulse transients can be generated with the aid of fiber optics. Fiber optic delivery systems are particularly maneuverable and can be used to irradiate target materials located adjacent to epithelial tissue layers to generate impulse transients in hard-to reach places. These types of delivery systems, when optically coupled to lasers, are preferred as they can be integrated into catheters and related flexible devices, and used to irradiate most organs in the human body. In addition, to launch an impulse transient having the desired rise times and peak stress, the wavelength of the optical source can be easily tailored to generate the appropriate absorption in a particular target material.

Alternatively, an energetic material can produce an impulse transient in response to a detonating impulse. The detonator can detonate the energetic material by causing an electrical discharge or spark.

Hydrostatic pressure can be used in conjunction with impulse transients to enhance the transport of a compound through the epithelial tissue layer. Since the effects induced by the impulse transients last for several minutes, the transport rate of a drug diffusing passively through the epithelial cell layer along its concentration gradient can be increased by applying hydrostatic pressure on the surface of the epithelial tissue layer, e.g., the stratum corneum of the skin, following application of the impulse transient.

### Skewing of Developing TCR Repertoire Towards, or Away From, Specific Antigens

The ability to enhance the uptake into the thymus of hematopoietic stem cells means that the nature and type of dendritic cells can be manipulated. For example the stem cells could be transfected with specific gene(s) which eventually become expressed in the dendritic cells in the thymus (and elsewhere in the body). Such genes could include those which encode specific antigens for which an immune response would be detrimental, as in autoimmune diseases, allergies and non-autologous grafts. The genes could also encode antigens (also as peptides) for which an immune response is desired, e.g. tumor antigens and microorganisms. In the latter case the level and affinity of the peptide would be manipulated to be low enough not to induce negative selection but high enough to promote positive selection. See copending patent application U.S. Ser. No. 09/______, incorporated herein by reference. We have shown that positive selection can involve multiple cell types: the cortical epithelium provides the specific differentiation molecules and third party cells the MHC/peptide ligands.

The precursors can also be genetically modified at the level of soluble regulatory molecules such as chemokines, cytokines and other molecules affecting any aspect of thymopoiesis and T cell development, activation, positive or negative selection, migration, and general status. This approach can be used to promote or retard thymic development or T cell responsiveness. It can be used to skew the T cell repertoire to specific antigens such as anti-viral and anti-tumor defenses. This approach can also be used to modulate the nature, organization and function of the thymic microenvironment.

The most effective means of generating tolerance to self is through intra-thymic deletion (or anergy or induction of negative regulatory cells) of the potentially self reactive cells through negative selection, mediated most efficiently by intrathymic dendritic cells. As a corollary, the establishment of tolerance to self antigens could be best achieved if dendritic cells expressing this antigen could be incorporated into the thymus. This form of tolerance may also be made more effective through the advent of inhibitory immunoregulatory cells. The mechanisms underlying the development of the latter, however, are poorly understood, but also occur in the thymus and could involve dendritic cells.

In the case of hyperreactive T cells for which the target antigen is known, hematopoietic stem cells (HSC) and/or epithelial stem cells (preferably autologous) can be transfected with the gene encoding the specific antigen for which tolerance is desired. When these cells develop into dendritic cells in the thymus they delete any new T cells arising which are potentially reactive to the nominal antigen.

While not required, the additional HSC can speed the return of the patient's immune system to a fully active state. Further, if the diseased patient's HSC or T cell progenitors are abnormal, replacement by the added HSC will remove or vastly deplete the abnormal cells.

The enormous clinical benefits to be gained through restoration of thymic function represent an important strategy for the treatment of immunodeficiencies, particularly in the elderly, HIV patients and following chemotherapy. Furthermore patients who have functionally abnormal T cells can now be treated to remove all T cells, thereby stopping the disease, and then have their normal immunity restored by reactivation of thymic function by inhibition of sex steroid production. SMALL ANIMAL STUDIES

### Materials and Methods

### Animals

CBA/CAH and C57B16/J male mice were obtained from Central Animal Services, Monash University and were housed under conventional conditions. Ages ranged from 4-6 weeks to 26 months of age and are indicated where relevant.

### Castration

Animals were anesthetized by intraperitoneal injection of 0.3 ml of 0.3 mg xylazine (Rompun; Bayer Australia Ltd., Botany NSW, Australia) and 1.5 mg ketamine hydrochloride (Ketalar; Parke-Davis, Caringbah, NSW, Australia) in saline. Surgical castration was performed by a scrotal incision, revealing the testes, which were tied with suture and then removed along with surrounding fatty tissue.

### Bromodeoxyuridine (BrdU) Incorporation

Mice received two intraperitoneal injections of BrdU (Sigma Chemical Co., St. Louis, Mo.) (100 mg/kg body weight in 100 μl of PBS) at a 4 hour interval. Control mice received vehicle alone injections. One hour after the second injection, thymuses were dissected and either a cell suspension made for FACS analysis, or immediately embedded in Tissue Tek (O.C.T. compound, Miles INC, Indiana), snap frozen in liquid nitrogen, and stored at −70° C. until use.

### Flow Cytometric Analysis

Mice were killed by CO₂ asphyxiation and thymus, spleen and mesenteric lymph nodes were removed. Organs were pushed gently through a 200 μm sieve in cold PBS/1% FCS/0.02% Azide, centrifuged (650 g, 5 min, 4° C.), and resuspended in either PBS/FCS/Az. Spleen cells were incubated in red cell lysis buffer (8.9 g/liter ammonium chloride) for 10 min at 4° C., washed and resuspended in PBS/FCS/Az. Cell concentration and viability were determined in duplicate using a hemocytometer and ethidium bromide/acridine orange and viewed under a fluorescence microscope (Axioskop; Carl Zeiss, Oberkochen, Germany).

For 3-color immunofluorescence thymocytes were routinely labeled with anti-αβTCR-FITC or anti-γδTCR-FITC, anti-CD4-PE and anti-CD8-APC (all obtained from Pharmingen, San Diego, Calif.) followed by flow cytometry analysis. Spleen and lymph node suspensions were labeled with either αβTCR-FITC/CD4-PE/CD8-APC or B220-B (Sigma) with CD4-PE and CD8-APC. B220-B was revealed with streptavidin-Tri-color conjugate purchased from Caltag Laboratories, Inc., Burlingame, Calif.

For BrdU detection, cells were surface labeled with CD4-PE and CD8-APC, followed by fixation and permeabilization as previously described (Carayon and Bord, 1989). Briefly, stained cells were fixed O/N at 4° C. in 1% PFA/0.01% Tween-20. Washed cells were incubated in 500 μl DNase (100 Kunitz units, Boehringer Mannheim, W. Germany) for 30 mins at 37° C. in order to denature the DNA. Finally, cells were incubated with anti-BrdU-FITC (Becton-Dickinson).

For 4-color Immunofluorescence thymocytes were labeled for CD3, CD4, CD8, B220 and Mac-1, collectively detected by anti-rat Ig-Cy5 (Amersham, U.K.), and the negative cells (TN) gated for analysis. They were further stained for CD25-PE (Pharmingen) and CD44-B (Pharmingen) followed by Streptavidin-Tri-colour (Caltag, CA) as previously described (Godfrey and Zlotnik, 1993). BrdU detection was then performed as described above.

Samples were analyzed on a FacsCalibur (Becton-Dickinson). Viable lymphocytes were gated according to 0° and 90° light scatter profiles and data was analyzed using Cell quest software (Becton-Dickinson).

### Immunohistology

Frozen thymus sections (4 μm) were cut using a cryostat (Leica) and immediately fixed in 100% acetone.

For two-color immunofluorescence, sections were double-labeled with a panel of monoclonal antibodies: MTS6, 10, 12, 15, 16, 20, 24, 32, 33, 35 and 44 (Godfrey et al., 1990; Table 1) produced in this laboratory and the co-expression of epithelial cell determinants was assessed with a polyvalent rabbit anti-cytokeratin Ab (Dako, Carpinteria, Calif.). Bound mAb was revealed with FITC-conjugated sheep anti-rat Ig (Silenus Laboratories) and anti-cytokeratin was revealed with TRITC-conjugated goat anti-rabbit Ig (Silenus Laboratories).

For BrdU detection, sections were stained with either anti-cytokeratin followed by anti-rabbit-TRITC or a specific mAb, which was then revealed with anti-rat Ig-Cγ3 (Amersham). BrdU detection was then performed as previously described (Penit et al., 1996). Briefly, sections were fixed in 70% Ethanol for 30 mins. Semi-dried sections were incubated in 4M HCl, neutralized by washing in Borate Buffer (Sigma), followed by two washes in PBS. BrdU was detected using anti-BrdU-FITC (Becton-Dickinson).

For three-color immunofluorescence, sections were labeled for a specific MTS mAb together with anti-cytokeratin. BrdU detection was then performed as described above.

Sections were analyzed using a Leica fluorescent and Nikon confocal microscopes.

### Migration Studies

Animals were anesthetized by intraperitoneal injection of 0.3 ml of 0.3 mg xylazine (Rompun; Bayer Australia Ltd., Botany NSW, Australia) and 1.5 mg ketamine hydrochloride (Ketalar; Parke-Davis, Caringbah, NSW, Australia) in saline.

Details of the FITC labeling of thymocytes technique are similar to those described elsewhere (Scollay et al., 1980; Berzins et al., 1998). Briefly, thymic lobes were exposed and each lobe was injected with approximately 10 μm of 350 μg/ml FITC (in PBS). The wound was closed with a surgical staple, and the mouse was warmed until fully recovered from anaesthesia. Mice were killed by CO₂ asphyxiation approximately 24 h after injection and lymphoid organs were removed for analysis.

After cell counts, samples were stained with anti-CD4-PE and anti-CD8-APC, then analyzed by flow cytometry. Migrant cells were identified as live-gated FITC⁺ cells expressing either CD4 or CD8 (to omit autofluorescing cells and doublets). The percentages of FITC⁺ CD4 and CD8 cells were added to provide the total migrant percentage for lymph nodes and spleen, respectively. Calculation of daily export rates was performed as described by Berzins et al. (1998).

Data analyzed using the unpaired student ‘t’ test or nonparametrical Mann-Whitney test was used to determine the statistical significance between control and test results for experiments performed at least in triplicate. Experimental values significantly differing from control values are indicated as follows: *p≦0.05, **p≦0.01 and ***p≦0.001.

### Results

### The Effect of Age on Thymocyte Populations

(i) Thymic Weight and Thymocyte Number

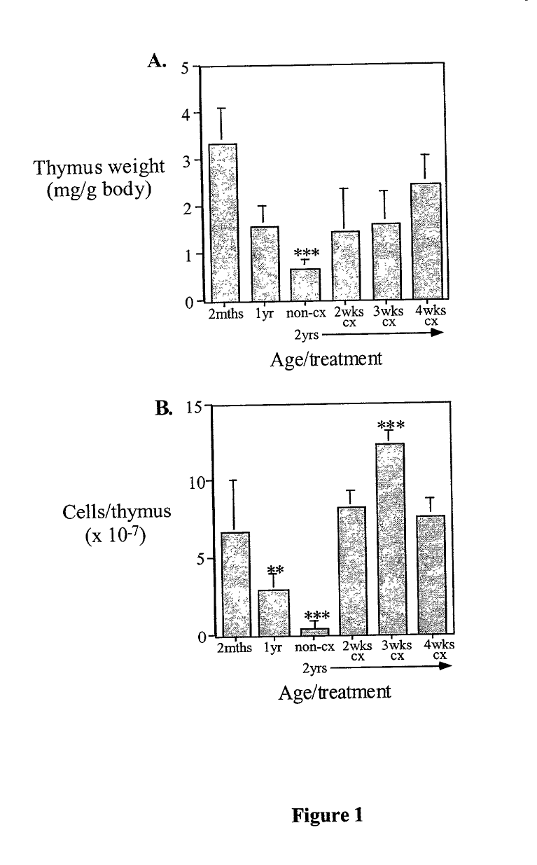

With increasing age there is a highly significant (p≦0.0001) decrease in both thymic weight (FIG. 1A) and total thymocyte number (FIG. 1B). Relative thymic weight (mg thymus/g body) in the young adult has a mean value of 3.34 which decreases to 0.66 at 18-24 months of age (adipose deposition limits accurate calculation). The decrease in thymic weight can be attributed to a decrease in total thymocyte numbers: the 1-2 month thymus contains ˜6.7×1 thymocytes, decreasing to ˜4.5×10⁶ cells by 24 months. By removing the effects of sex steroids on the thymus by castration, regeneration occurs and by 4 weeks post-castration, the thymus is equivalent to that of the young adult in both weight and cellularity (FIGS. 1A and 1B). Interestingly, there is a significant (p≦0.001) increase in thymocyte numbers at 2 weeks post-castration (˜1.2×10⁸), which is restored to normal young levels by 4 weeks post-castration (FIG. 1B).

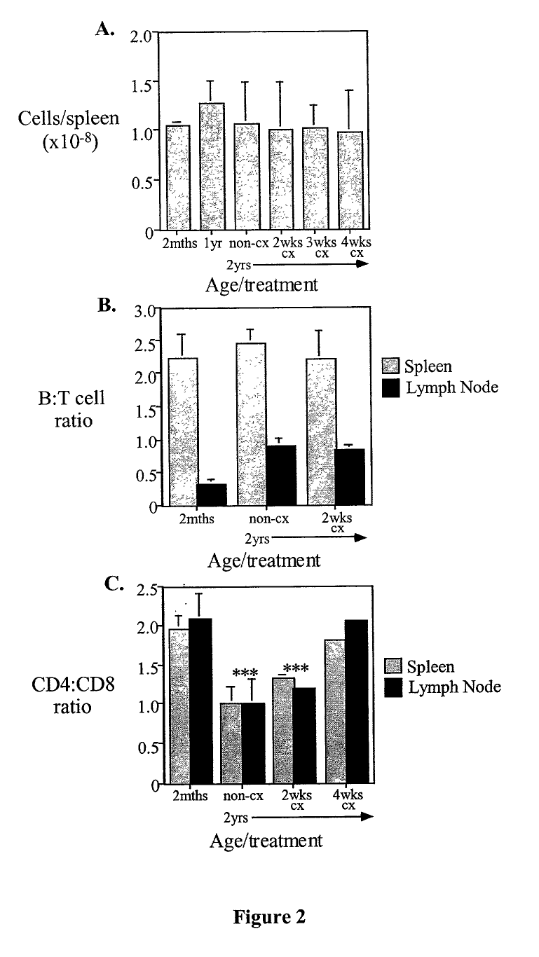

The decrease in T cell numbers produced by the thymus is not reflected in the periphery, with spleen cell numbers remaining constant with age (FIG. 2A). Homeostatic mechanisms in the periphery were evident since the B cell to T cell ratio in spleen and lymph nodes was not affected with age and the subsequent decrease in T cell numbers reaching the periphery (FIG. 2B). However, the ratio of CD4⁺ to CD8⁺ T cell significantly decreased (p≦0.001) with age from 2:1 at 2 months of age, to a ratio of 1:1 at 2 years of age (FIG. 2C). Following castration and the subsequent rise in T cell numbers reaching the periphery, no change in peripheral T cell numbers was observed: splenic T cell numbers and the ratio of B:T cells in both spleen and lymph nodes was not altered following castration (FIG. 2A and B). The decreased CD4:CD8 ratio in the periphery with age was still evident at 2 weeks post-castration but was completely reversed by 4 weeks post-castration (FIG. 2C).

(ii) αβTCR, γδTCR, CD4 and CD8 expression

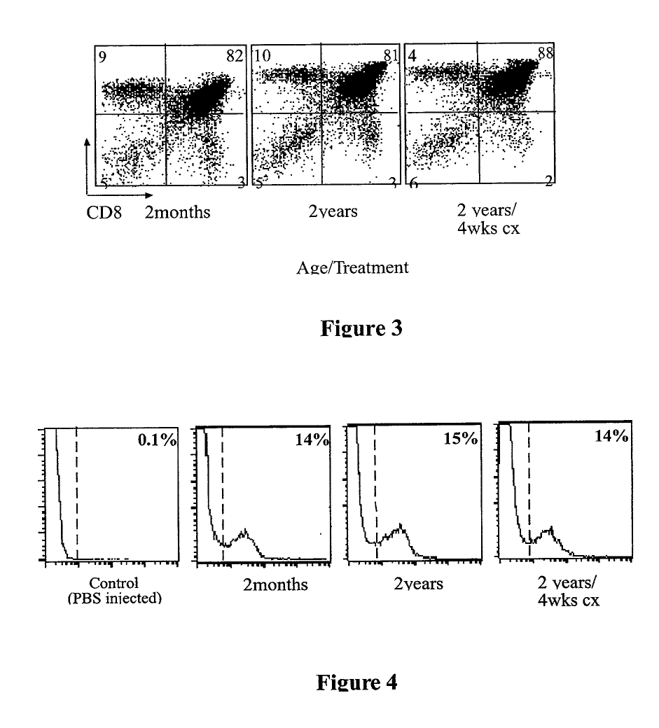

To determine if the decrease in thymocyte numbers seen with age was the result of the depletion of specific cell populations, thymocytes were labeled with defining markers in order to analyze the separate subpopulations. In addition, this allowed analysis of the kinetics of thymus repopulation post-castration. The proportion of the main thymocyte subpopulations was compared with those of the normal young thymus (FIG. 3) and found to remain uniform with age. In addition, further subdivision of thymocytes by the expression of αβTCR and γδTCR revealed no change in the proportions of these populations with age (data not shown). At 2 and 4 weeks post-castration, thymocyte subpopulations remained in the same proportions and, since thymocyte numbers increase by up to 100-fold post-castration, this indicates a synchronous expansion of all thymocyte subsets rather than a developmental progression of expansion.

The decrease in cell numbers seen in the thymus of aged animals thus appears to be the result of a balanced reduction in all cell phenotypes, with no significant changes in T cell populations being detected. Thymus regeneration occurs in a synchronous fashion, replenishing all T cell subpopulations simultaneously rather than sequentially.

### Proliferation of Thymocytes

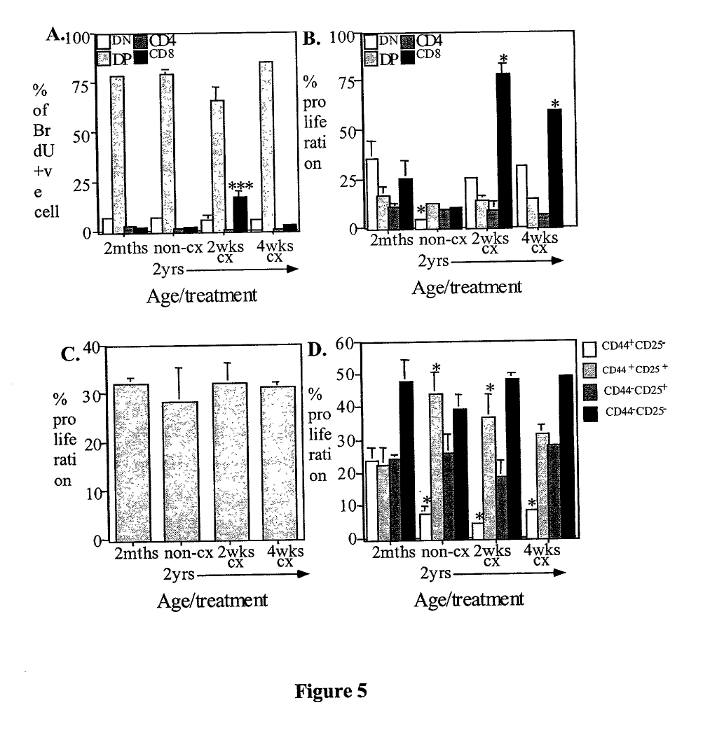

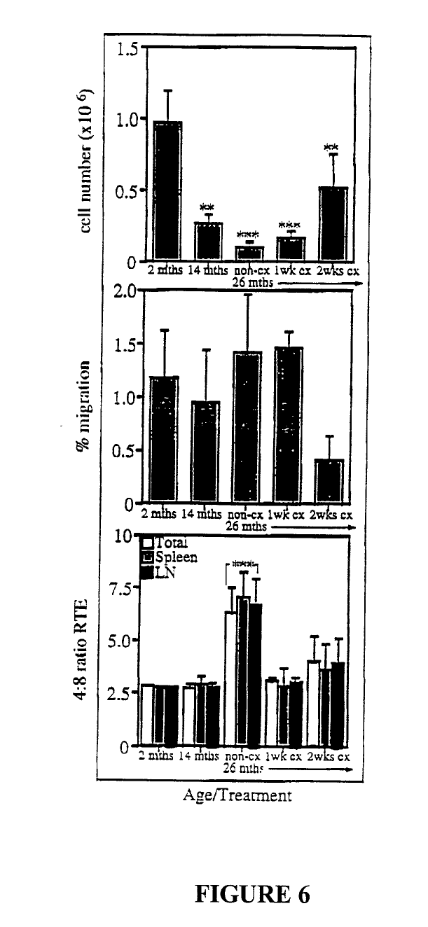

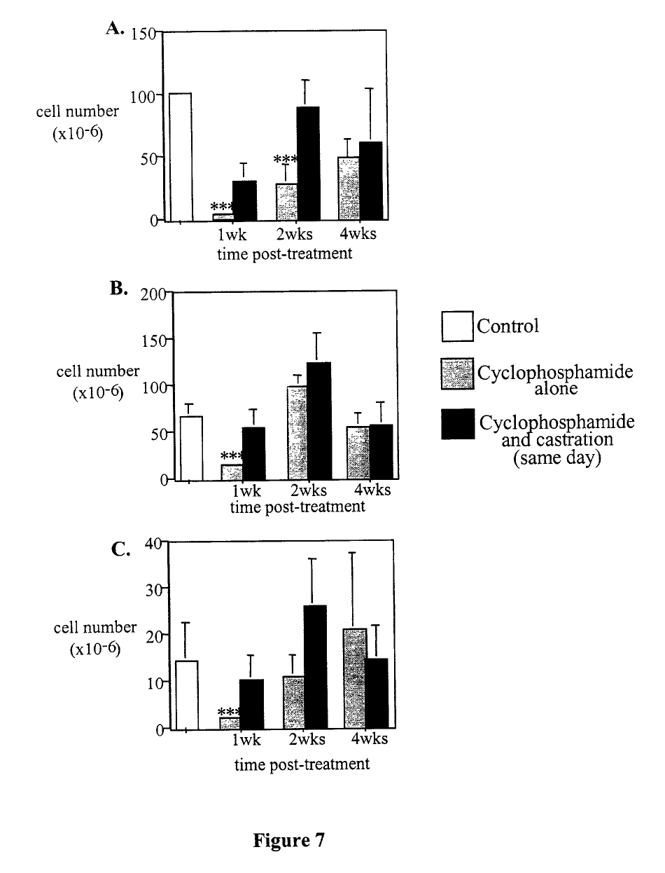

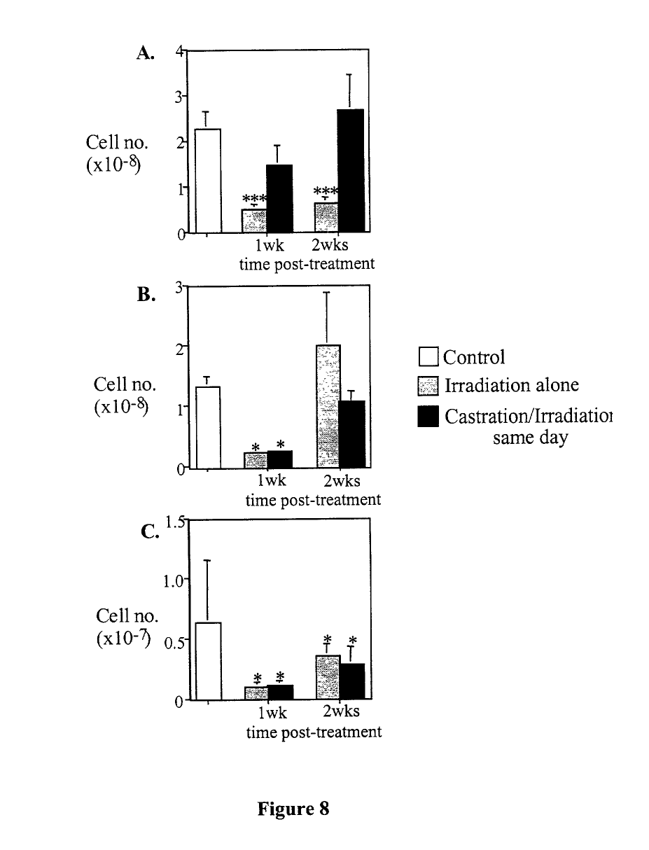

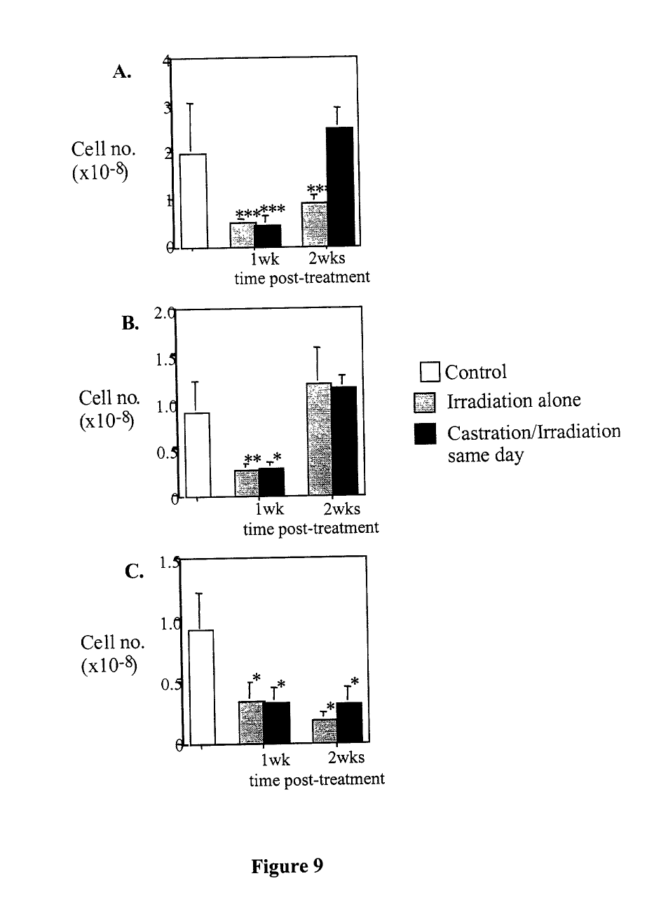

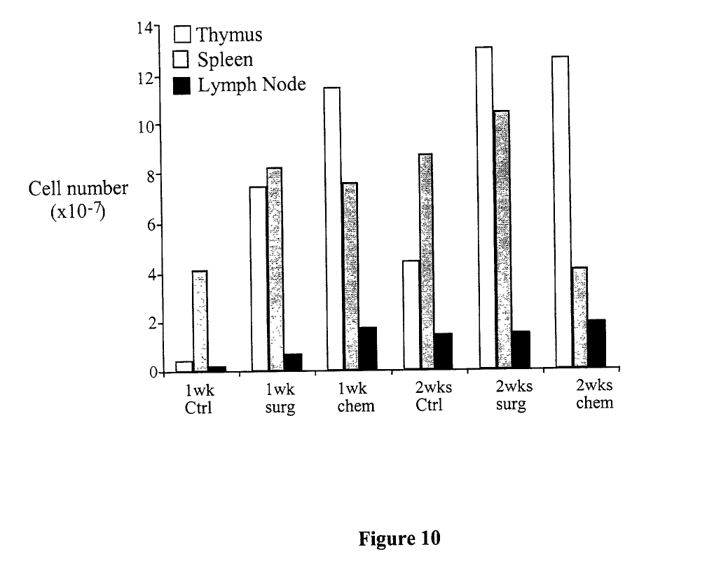

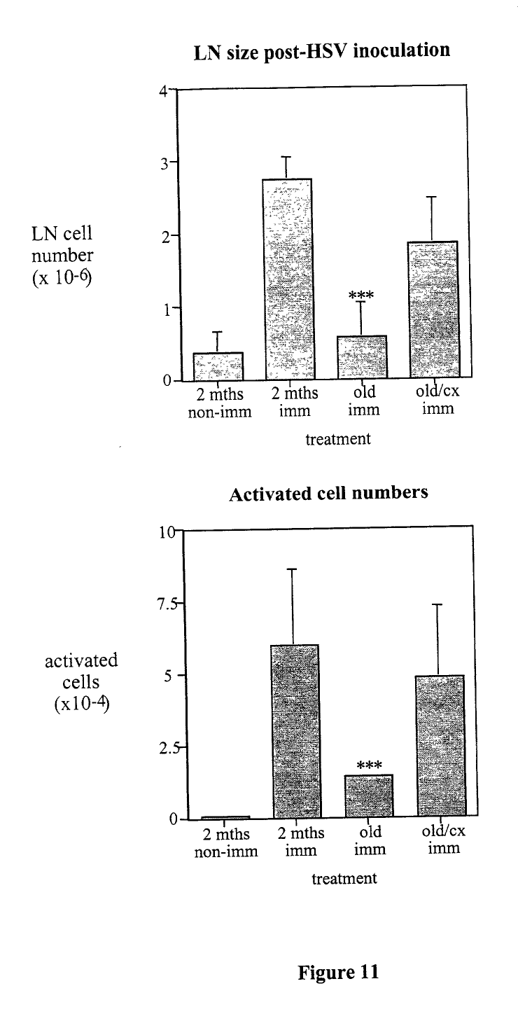

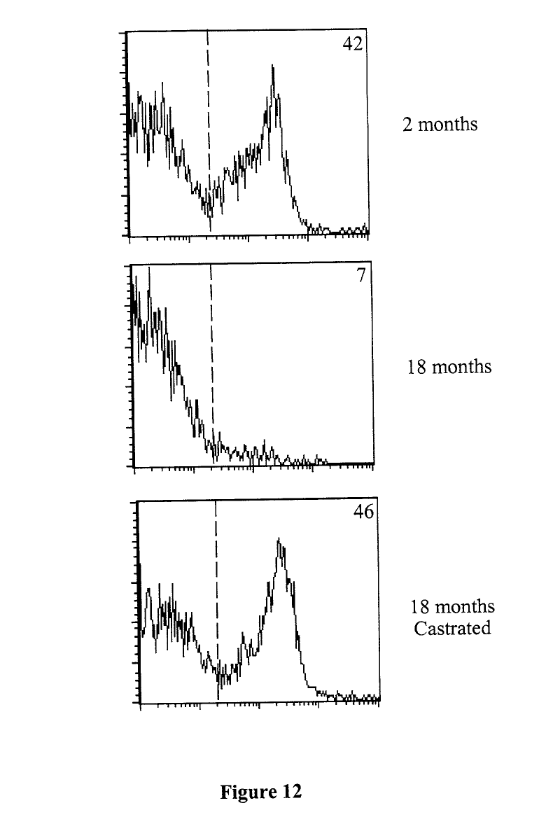

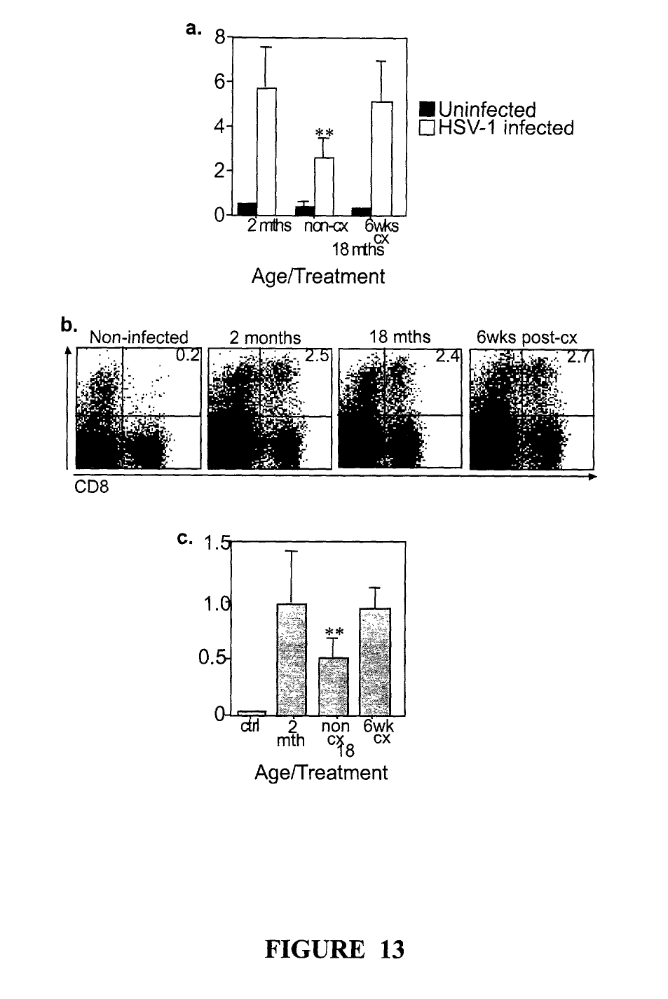

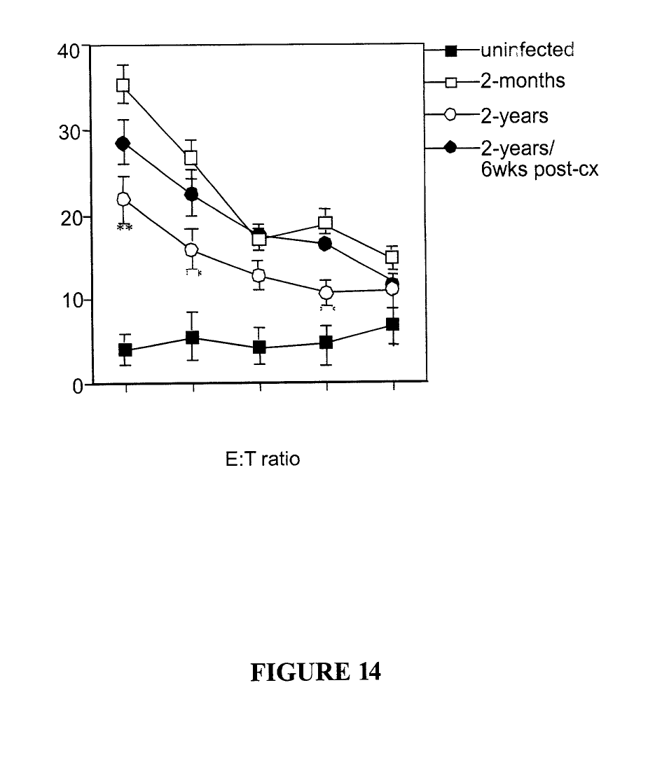

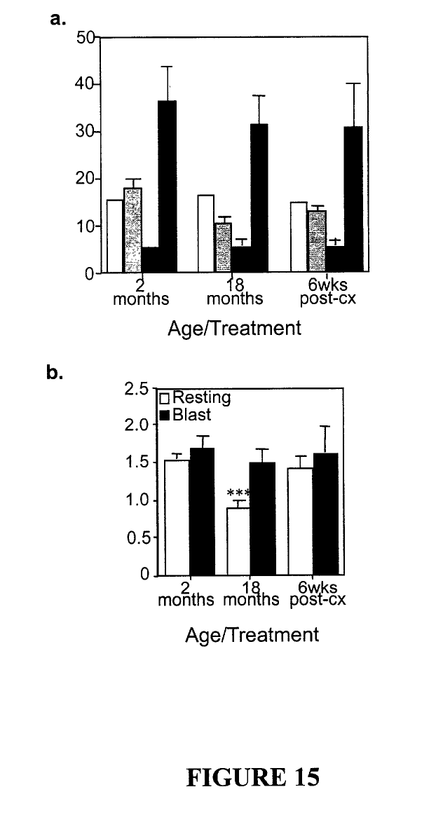

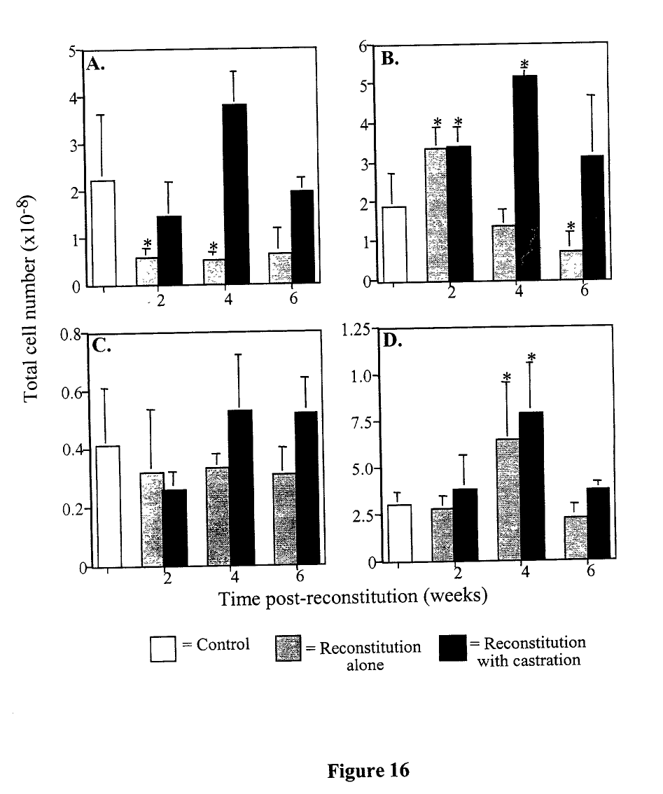

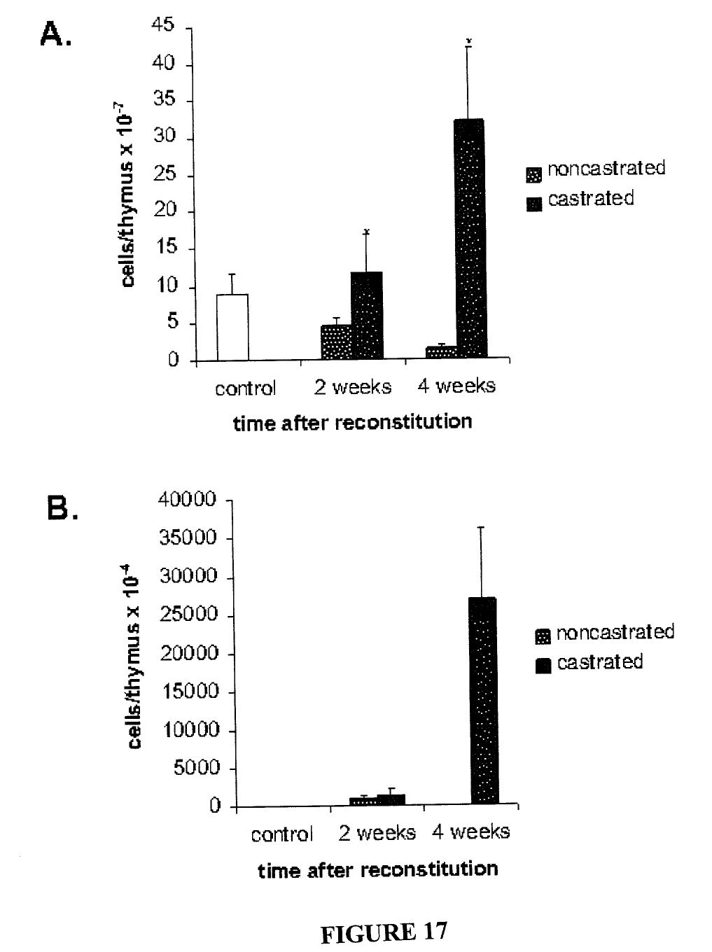

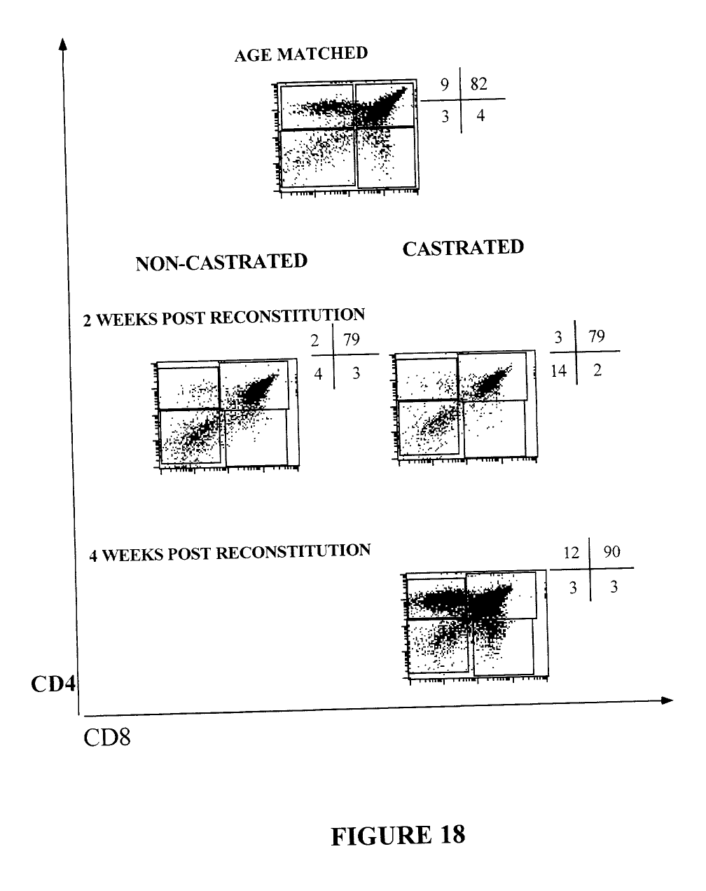

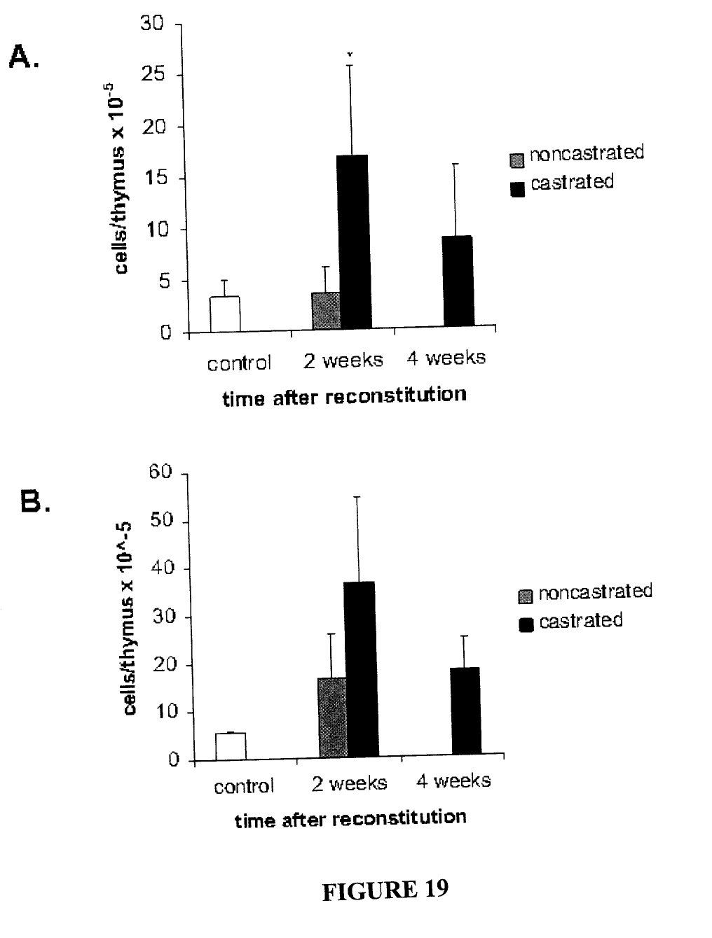

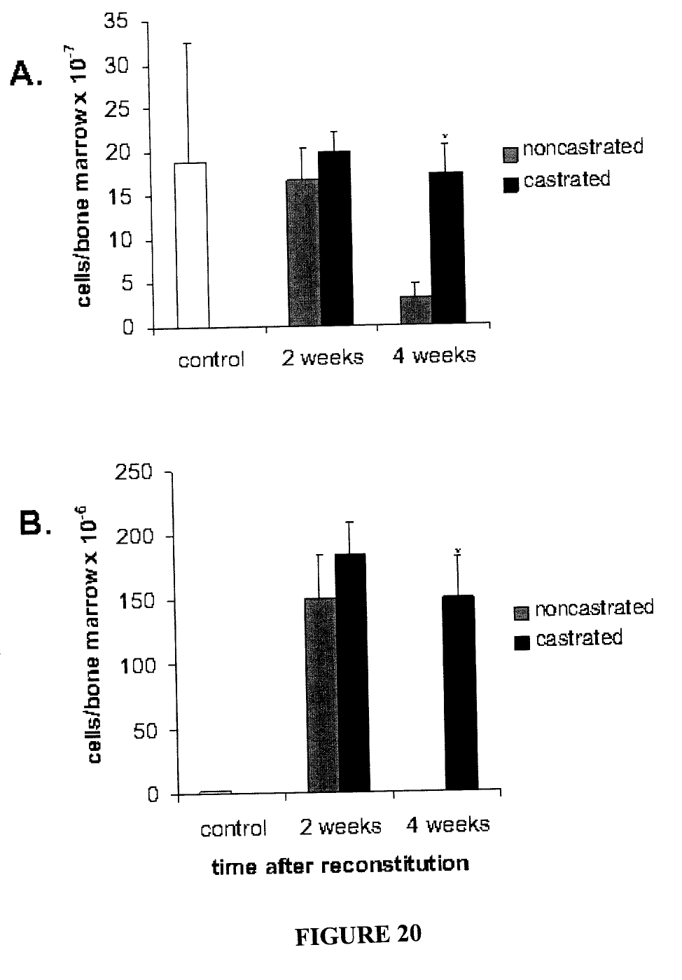

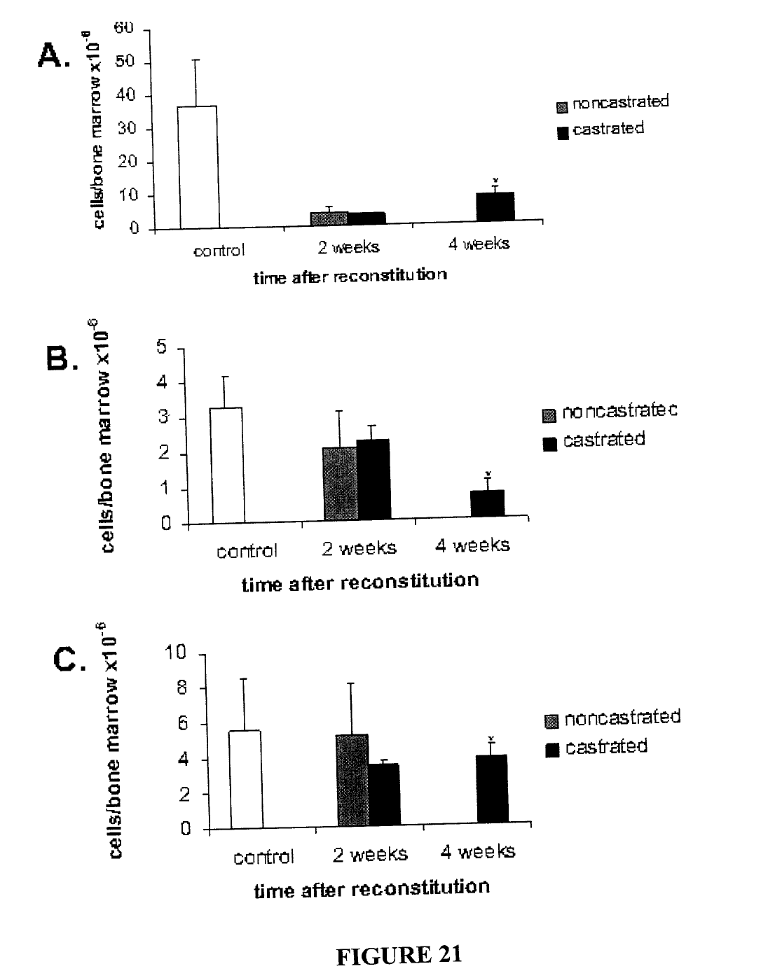

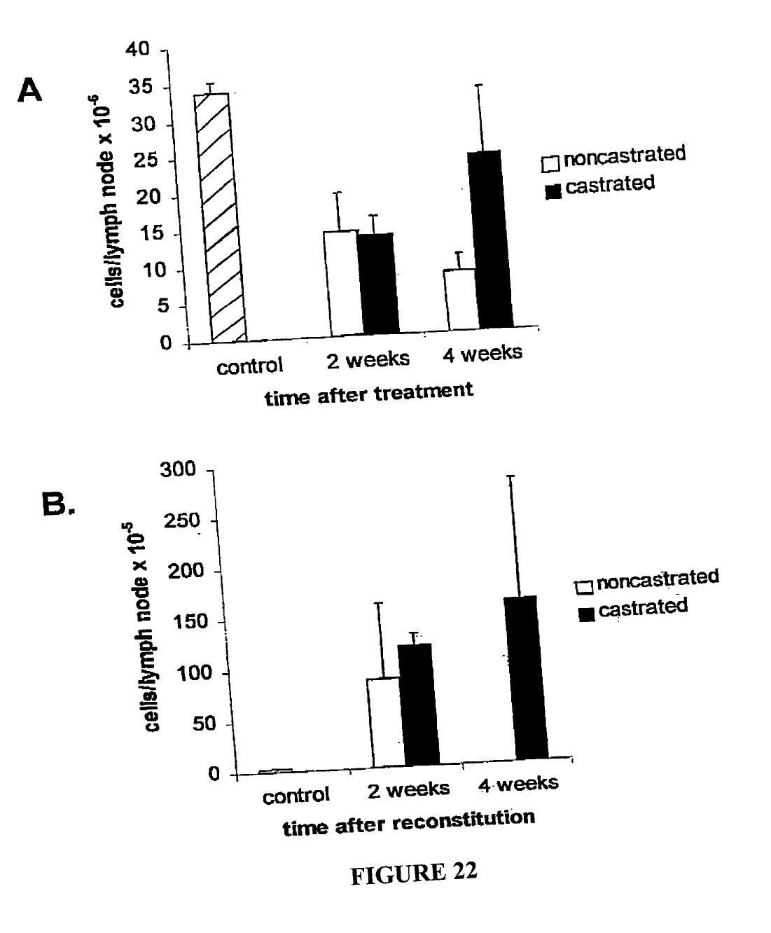

As shown in FIG. 4, 15-20% of thymocytes are proliferating at 4-6 weeks of age. The majority (˜80%) of these are DP with the TN subset making up the second largest population at ˜6% (FIG. 5A). Accordingly, most division is seen in the subcapsule and cortex by immunohistology (data not shown). Some division is seen in the medullary regions with FACS analysis revealing a proportion of SP cells (9% of CD4 T cells and 25% of CD8 T cells) dividing (FIG. 5B).

Although cell numbers are significantly decreased in the aged thymus, proliferation of thymocytes remains constant, decreasing to 12-15% at 2 years (FIG. 4), with the phenotype of the proliferating population resembling the 2 month thymus (FIG. 5A). Immunohistology revealed the division at 1 year of age to reflect that seen in the young adult; however, at 2 years, proliferation is mainly seen in the outer cortex and surrounding the vasculature (data not shown). At 2 weeks post-castration, although thymocyte numbers significantly increase, there is no change in the proportion of thymocytes that are proliferating, again indicating a synchronous expansion of cells (FIG. 4). Immunohistology revealed the localization of thymocyte proliferation and the extent of dividing cells to resemble the situation in the 2-month-old thymus by 2 weeks post-castration (data not shown). When analyzing the proportion of each subpopulation which represent the proliferating population, there was a significant (p<0.001) increase in the percentage of CD8 T cells which are within the proliferating population (1% at 2 months and 2 years of age, increasing to ˜6% at 2 weeks post-castration) (FIG. 5A).

FIG. 5B illustrates the extent of proliferation within each subset in young, old and castrated mice. There is a significant (p≦0.001) decay in proliferation within the DN subset (35% at 2 months to 4% by 2 years). Proliferation of CD8+ T cells was also significantly (p≦0.001) decreased, reflecting the findings by immunohistology (data not shown) where no division is evident in the medulla of the aged thymus. The decrease in DN proliferation is not returned to normal young levels by 4 weeks post-castration. However, proliferation within the CD8+ T cell subset is significantly (p≦0.001) increased at 2 weeks post-castration and is returning to normal young levels at 4 weeks post-castration.

The decrease in proliferation within the DN subset was analyzed further using the markers CD44 and CD25. The DN subpopulation, in addition to the thymocyte precursors, contains αβTCR+ CD4-CD8-thymocytes, which are thought to have downregulated both co-receptors at the transition to SP cells (Godfrey & Zlotnik, 1993). By gating on these mature cells, it was possible to analyze the true TN compartment (CD3⁻CD4⁻CD8⁻) and these showed no difference in their proliferation rates with age or following castration (FIG. 5C). However, analysis of the subpopulations expressing CD44 and CD25, showed a significant (p<0.001) decrease in proliferation of the TN1 subset (CD44⁺CD25⁻), from 20% in the normal young to around 6% at 18 months of age (FIG. 5D) which was restored by 4 weeks post-castration. The decrease in the proliferation of the TN1 subset, was compensated for by a significant (p≦0.001) increase in proliferation of the TN2 subpopulation (CD44⁺CD25⁺) which returned to normal young levels by 2 weeks post-castration (FIG. 5D).

### The Effect of Age on the Thymic Microenvironment

The changes in the thymic microenvironment with age were examined by immunofluorescence using an extensive panel of MAbs from the MTS series, double-labeled with a polyclonal anti-cytokeratin Ab.

The antigens recognized by these MAbs can be subdivided into three groups: thymic epithelial subsets, vascular-associated antigens and those present on both stromal cells and thymocytes.

(i) Epithelial Cell Antigens

Anti-keratin staining (pan-epithelium) of 2 year old mouse thymus, revealed a loss of general thymus architecture with a severe epithelial cell disorganization and absence of a distinct cortico-medullary junction. Further analysis using the MAbs, MTS 10 (medulla) and MTS44 (cortex), showed a distinct reduction in cortex size with age, with a less substantial decrease in medullary epithelium (data not shown). Epithelial cell free regions, or keratin negative areas (KNA's, van Ewijk et al., 1980; Godfrey et al., 1990; Bruijntjes et al, 1993).) were more apparent and increased in size in the aged thymus, as evident with anti-cytokeratin labeling. There is also the appearance of thymic epithelial “cyst-like” structures in the aged thymus particularly noticeable in medullary regions (data not shown). Adipose deposition, severe decrease in thymic size and the decline in integrity of the cortico-medullary junction are shown conclusively with the anti-cytokeratin staining (data not shown). The thymus is beginning to regenerate by 2 weeks post-castration. This is evident in the size of the thymic lobes, the increase in cortical epithelium as revealed by MTS 44, and the localization of medullary epithelium. The medullary epithelium is detected by MTS 10 and at 2 weeks, there are still subpockets of epithelium stained by MTS 10 scattered throughout the cortex. By 4 weeks post-castration, there is a distinct medulla and cortex and discernible cortico-medullary junction (data not shown).

The markers MTS 20 and 24 are presumed to detect primordial epithelial cells (Godfrey, et al., 1990) and further illustrate the degeneration of the aged thymus. These are present in abundance at E14, detect isolated medullary epithelial cell clusters at 4-6 weeks but are again increased in intensity in the aged thymus (data not shown). Following castration, all these antigens are expressed at a level equivalent to that of the young adult thymus (data not shown) with MTS 20 and MTS 24 reverting to discrete subpockets of epithelium located at the cortico-medullary junction.

(ii) Vascular-associated Antigens

The blood-thymus barrier is thought to be responsible for the immigration of T cell precursors to the thymus and the emigration of mature T cells from the thymus to the periphery.

The MAb MTS 15 is specific for the endothelium of thymic blood vessels, demonstrating a granular, diffuse staining pattern (Godfrey, et al, 1990). In the aged thymus, MTS 15 expression is greatly increased, and reflects the increased frequency and size of blood vessels and perivascular spaces (data not shown).

The thymic extracellular matrix, containing important structural and cellular adhesion molecules such as collagen, laminin and fibrinogen, is detected by the mAb MTS 16. Scattered throughout the normal young thymus, the nature of MTS 16 expression becomes more widespread and interconnected in the aged thymus. Expression of MTS 16 is increased further at 2 weeks post-castration while 4 weeks post-castration, this expression is representative of the situation in the 2 month thymus (data not shown).

(iii) Shared Antigens

MHC II expression in the normal young thymus, detected by the MAb MTS 6, is strongly positive (granular) on the cortical epithelium (Godfrey et al., 1990) with weaker staining of the medullary epithelium. The aged thymus shows a decrease in MHC II expression with expression substantially increased at 2 weeks post-castration. By 4 weeks post-castration, expression is again reduced and appears similar to the 2 month old thymus (data not shown).

### Thymocyte Emigration

Approximately 1% of T cells migrate from the thymus daily in the young mouse (Scollay et al., 1980). We found migration was occurring at a proportional rate equivalent to the normal young mouse at 14 months and even 2 years of age (FIG. 5) although significantly (p≦0.0001) reduced in number. There was an increase in the CD4:CD8 ratio of the recent thymic emigrants from ˜3:1 at 2 months to ˜7:1 at 26 months. By 1 week post-castration, cell number migrating to the periphery has substantially increased with the overall rate of migration remaining constant at 1-1.5%.

### EXAMPLES

The following Examples provide specific examples of methods of the invention, and are not to be construed as limiting the invention to their content. For convenience these examples describe delivery of an LHRH agonist to block sex steroid mediated signaling to the thymus. However, the scope of the invention is not so limited, as described above.

### Example 1

### T Cell Depletion

In order to remove the abnormal T cells, the patient underwent T cell depletion. One standard procedure for this step is as follows: The human patient received anti-T cell antibodies in the form of a daily injection of 15 mg/kg of Atgam (xeno anti-T cell globulin, Pharmacia Upjohn) for a period of 10 days in combination with an inhibitor of T cell activation, cyclosporin A, 3 mg/kg, as a continuous infusion for 3-4 weeks followed by daily tablets at 9 mg/kg as needed. This treatment did not affect early T cell development in the patient's thymus, as the amount of antibody necessary to have such an affect cannot be delivered due to the size and configuration of the human thymus. The treatment was maintained for approximately 4-6 weeks to allow the loss of sex steroids followed by the reconstitution of the thymus. The prevention of T cell reactivity may also be combined with inhibitors of second level signals such as interleukins or cell adhesion molecules to enhance the T cell ablation.

Because in many cases it is not possible to reduce only the antigen-specific T cells which cause the disease, the whole population of T cells, including the pathological ones, is depleted. This depletion of peripheral T cells markedly retards the disease. Simultaneously, however, because of the lack of T cells, it induces a state of generalized immunodeficiency which means the patients are highly susceptible to infection, particularly viral. Even B cell responses will not function normally in the absence of appropriate T cell help.

### Example 2

### Sex Steroid Ablation Therapy

The patient was given sex steroid ablation therapy in the form of delivery of an LHRH agonist. This was given in the form of either Leucrin (depot injection; 22.5 mg) or Zoladex (implant; 10.8 mg), either one as a single dose effective for 3 months. This was effective in reducing sex steroid levels sufficiently to reactivate the thymus. In some cases it is also necessary to deliver a suppresser of adrenal gland production of sex steroids, such as Cosudex (5 mg/day) as one tablet per day for the duration of the sex steroid ablation therapy. Adrenal gland production of sex steroids makes up around 10-15% of a human's steroids.

Reduction of sex steroids in the blood to minimal values took about 1-3 weeks; concordant with this was the reactivation of the thymus. In some cases it is necessary to extend the treatment to a second 3 month injection/implant.

### Example 3

### Alternative Delivery Method

In place of the 3 month depot or implant administration of the LHRH agonist, alternative methods can be used. In one example the patient's skin may be irradiated by a laser such as an Er:YAG laser, to ablate or alter the skin so as to reduce the impeding effect of the stratum corneum.

A. Laser Ablation or Alteration: An infrared laser radiation pulse was formed using a solid state, pulsed, Er:YAG laser consisting of two flat resonator mirrors, an Er:YAG crystal as an active medium, a power supply, and a means of focusing the laser beam. The wavelength of the laser beam was 2.94 microns. Single pulses were used.

The operating parameters were as follows: The energy per pulse was 40, 80 or 120 mJ, with the size of the beam at the focal point being 2 mm, creating an energy fluence of 1.27, 2.55 or 3.82 J/cm². The pulse temporal width was 300 μs, creating an energy fluence rate of 0.42, 0.85 or 1.27×10⁴ W/cm².

Subsequently, an amount of LHRH agonist is applied to the skin and spread over the irradiation site. The LHRH agonist may be in the form of an ointment so that it remains on the site of irradiation. Optionally, an occlusive patch is placed over the agonist in order to keep it in place over the irradiation site.

Optionally a beam splitter is employed to split the laser beam and create multiple sites of ablation or alteration. This provides a faster flow of LHRH agonist through the skin into the blood stream. The number of sites can be predetermined to allow for maintenance of the agonist within the patient's system for the requisite period of time.

B. Pressure Wave: A dose of LHRH agonist is placed on the skin in a suitable container, such as a plastic flexible washer (about 1 inch in diameter and about 1/16 inch thick), at the site where the pressure wave is to be created. The site is then covered with target material such as a black polystyrene sheet about 1 mm thick. A Q-switched solid state ruby laser (20 ns pulse duration, capable of generating up to 2 joules per pulse) is used to generate the laser beam, which hits the target material and generates a single impulse transient. The black polystyrene target completely absorbs the laser radiation so that the skin is exposed only to the impulse transient, and not to laser radiation. No pain is produced from this procedure. The procedure can be repeated daily, or as often as required, to maintain the circulating blood levels of the agonist.

### Example 4

### Optional Administration of HSC

In a preferred embodiment hematopoietic stem cells (HSC) are given to the patient. These are preferably autologous, but HSC from a mismatched donor can also be used. Where practical, the level of HSC in the patient's or donor's blood is enhanced by injecting the patient or donor with granulocyte-colony stimulating factor (G-CSF) at 10 Tg/kg for 2-5 days prior to cell collection. CD34⁺ cells are purified from the patient's or a donor's blood or bone marrow, preferably using a flow cytometer or immunomagnetic beading. HSC are identified by flow cytometry as being CD34⁺. Optionally these HSC are expanded ex vivo with Stem Cell Factor. At approximately 1-3 weeks post LHRH agonist delivery, just before or at the time the thymus begins to regenerate, the patient is injected with the HSC, optimally at a dose of about 24×10⁶ cells/kg. Optionally G-CSF may also be injected into the recipient to assist in expansion of the HSC.

The reactivated thymus takes up the purified HSC and converts them into new T cells. In the event that unmatched donor HSC are used, the donor dendritic cells will tolerize any T cells that are potentially reactive with the patient by inducing deletion by cell death, or by inducing tolerance through immunoregulatory cells.

Since the new T cells are purged of potentially self reactive and host reactive cells, having been positively selected by the host thymic epithelium, they are able to respond to normal infections by recognizing peptide presented by host APC in the periphery. Both patient and donor CD34+ HSC develop into dendritic cells, and subsequently into the patient's lymphoid system organs, and establish an immune system virtually identical to that of the patient alone, albeit with the appropriate corrections made. Thus normal immunoregulatory mechanisms will be present.

### Example 5

### Termination of Immunosuppression

When the thymic chimera is established and the new cohort of mature T cells have begun exiting the thymus, blood is taken from the patient and the status of T cells (and indeed all blood cells) is examined. In particular the T cells are examined for whether they are Th1 or Th2, and naïve or memory. In addition the types of cytokines they produce (Th1 versus Th2) are examined. Immunosuppressive therapy is gradually reduced to allow defense against infection. In the event that mismatched HSC have been administered, immunosuppressive therapy is stopped completely when there is no sign of rejection as indicated in part by the presence of activated T cells in the blood. Because the HSC have a strong self-renewal capacity, the hematopoietic chimera so formed will be stable, theoretically for the life of the patient (as for normal, non-tolerized people).

### Example 6

### Use of LHRH Agonist to Reactivate the Thymus in Humans

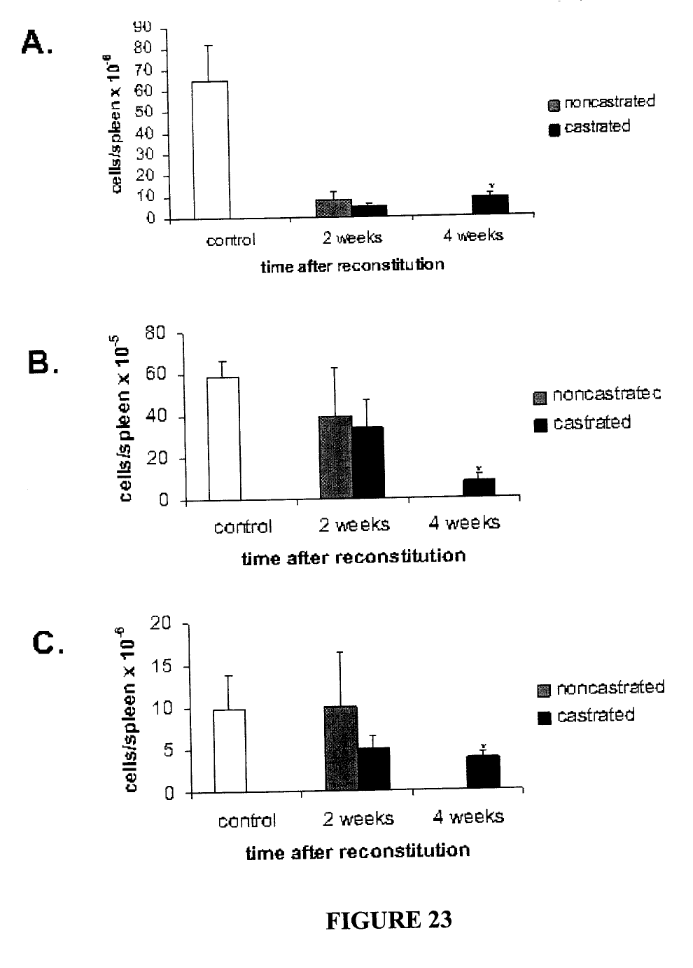

In order to show that a human thymus can be reactivated by the methods of this invention, these methods were used on patients who had been treated with chemotherapy for prostate cancer. Prostate cancer patients were evaluated before and 4 months after sex steroid ablation therapy. The results are summarized in FIGS. 23-27. Collectively the data demonstrate qualitative and quantitative improvement of the status of T cells in many patients.

The effect of LHRH therapy on total numbers of lymphocytes and T cells subsets thereof:

The phenotypic composition of peripheral blood lymphocytes was analyzed in patients (all>60 years) undergoing LHRH agonist treatment for prostate cancer (FIG. 23). Patient samples were analyzed before treatment and 4 months after beginning LHRH agonist treatment. Total lymphocyte cell numbers per ml of blood were at the lower end of control values before treatment in all patients. Following treatment, 6/9 patients showed substantial increases in total lymphocyte counts (in some cases a doubling of total cells was observed). Correlating with this was an increase in total T cell numbers in 6/9 patients. Within the CD4⁺ subset, this increase was even more pronounced with 8/9 patients demonstrating increased levels of CD4⁺ T cells. A less distinctive trend was seen within the CD8+ subset with 4/9 patients showing increased levels albeit generally to a smaller extent than CD4⁺ T cells.

The Effect of LHRH Therapy on the Proportion of T Cells Subsets

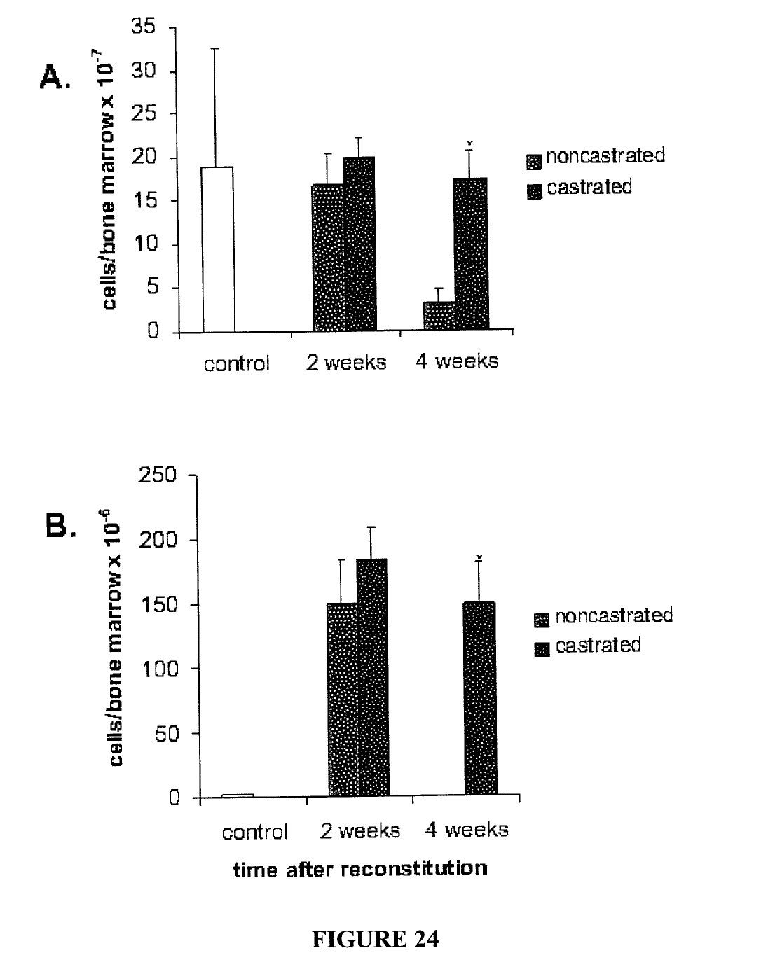

Analysis of patient blood before and after LHRH agonist treatment demonstrated no substantial changes in the overall proportion of T cells, CD4⁺ or CD8⁺ T cells and a variable change in the CD4⁺:CD8⁺ ratio following treatment (FIG. 24). This indicates that there was little effect of treatment on the homeostatic maintenance of T cell subsets despite the substantial increase in overall T cell numbers following treatment. All values were comparative to control values.

The Effect of LHRH Therapy on the Proportion of B Cells and Myeloid Cells

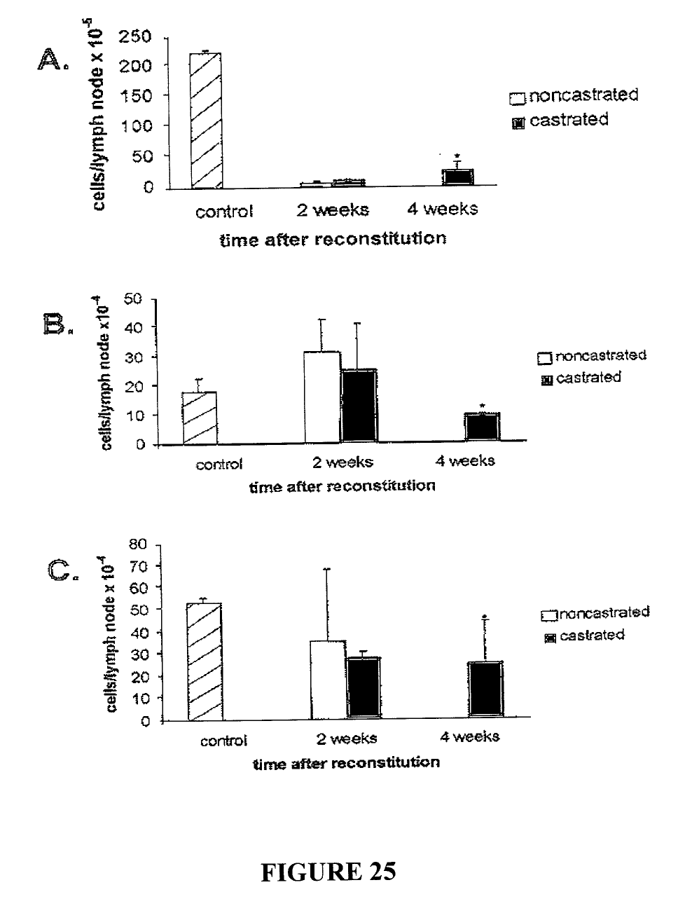

Analysis of the proportions of B cells and myeloid cells (NK, NKT and macrophages) within the peripheral blood of patients undergoing LHRH agonist treatment demonstrated a varying degree of change within subsets (FIG. 25). While NK, NKT and macrophage proportions remained relatively constant following treatment, the proportion of B cells was decreased in 4/9 patients.

The Effect of LHRH Agonist Therapy on the Total Number of B Cells and Myeloid Cells

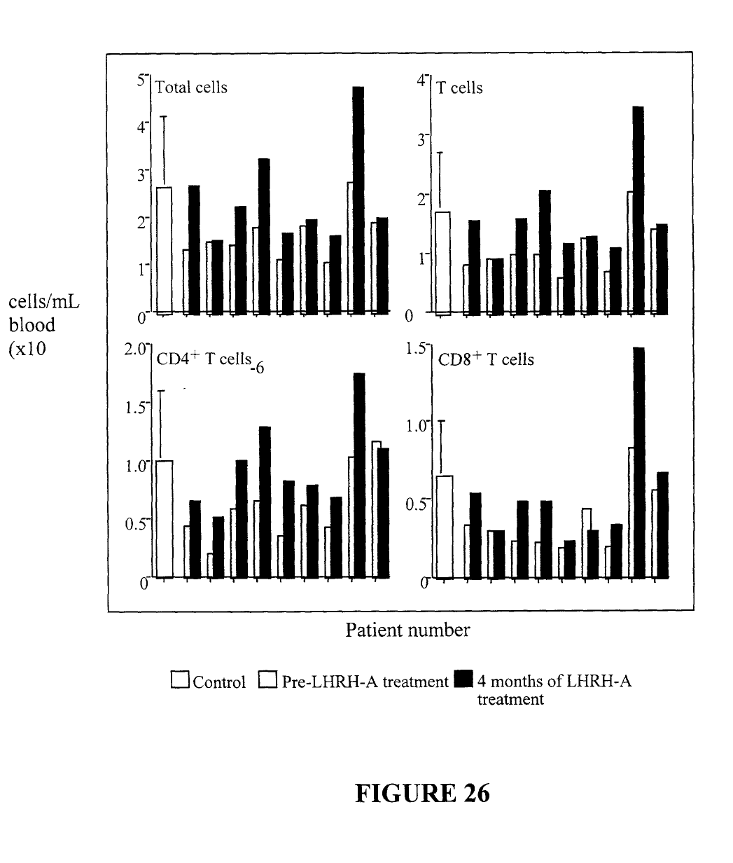

Analysis of the total cell numbers of B and myeloid cells within the peripheral blood post-treatment showed clearly increased levels of NK (5/9 patients), NKT (4/9 patients) and macrophage (3/9 patients) cell numbers post-treatment (FIG. 26). B cell numbers showed no distinct trend with 2/9 patients showing increased levels; 4/9 patients showing no change and 3/9 patients showing decreased levels.

The Effect of LHRH Therapy on the Level of Naive Cells Relative to Memory Cells

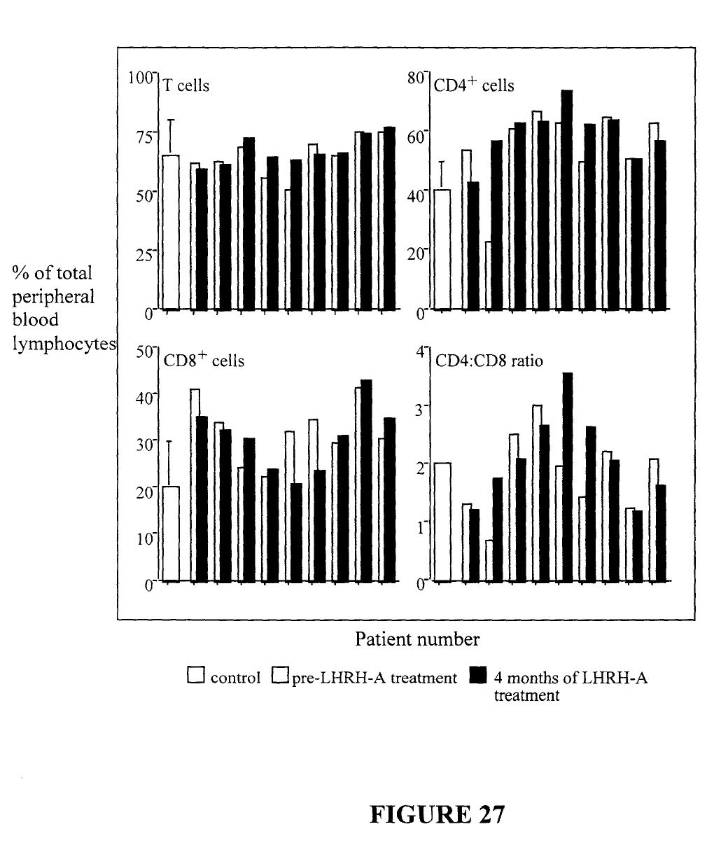

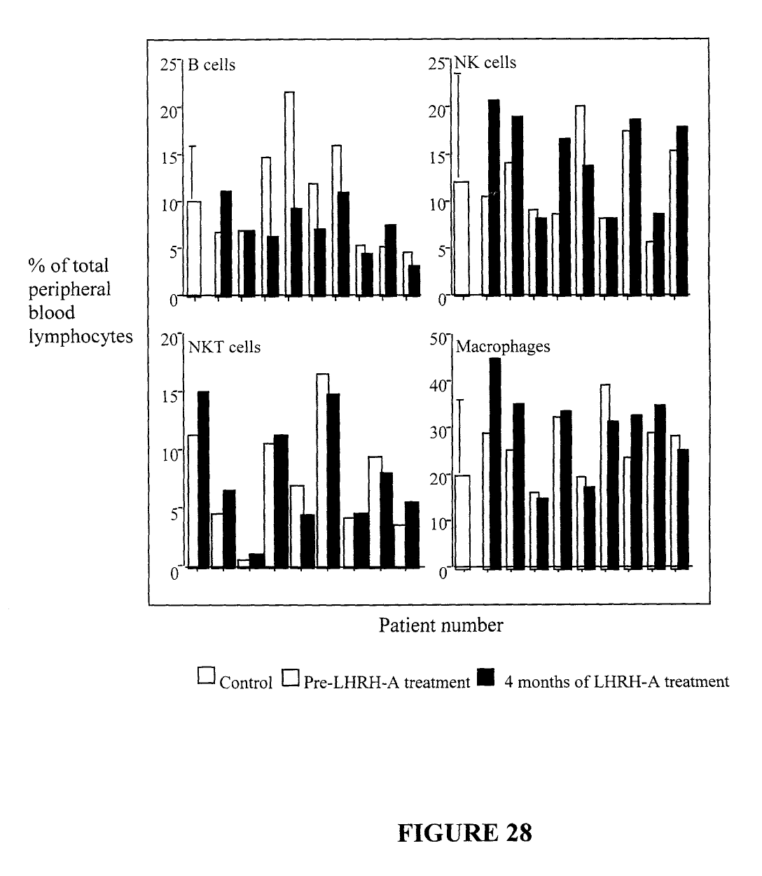

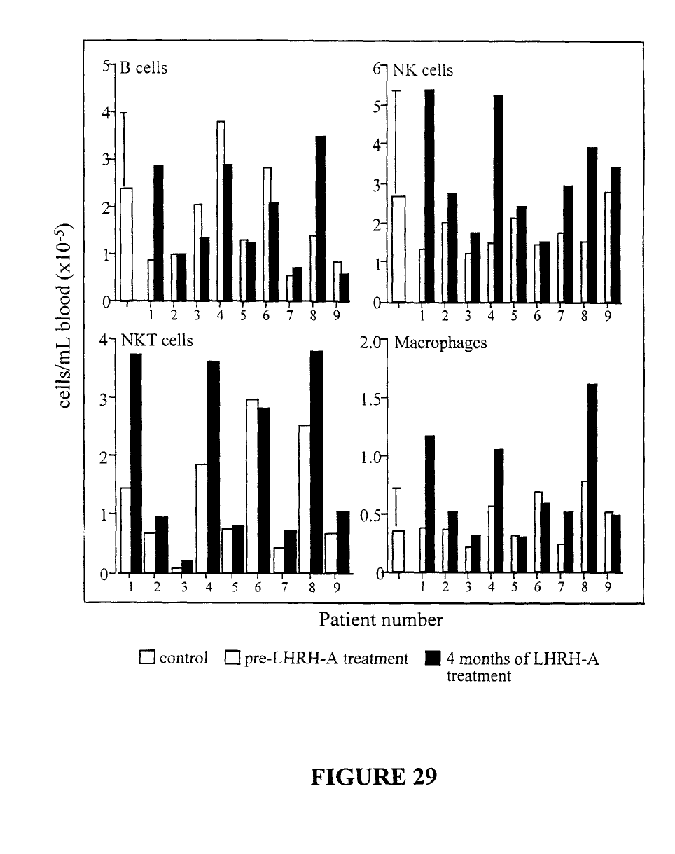

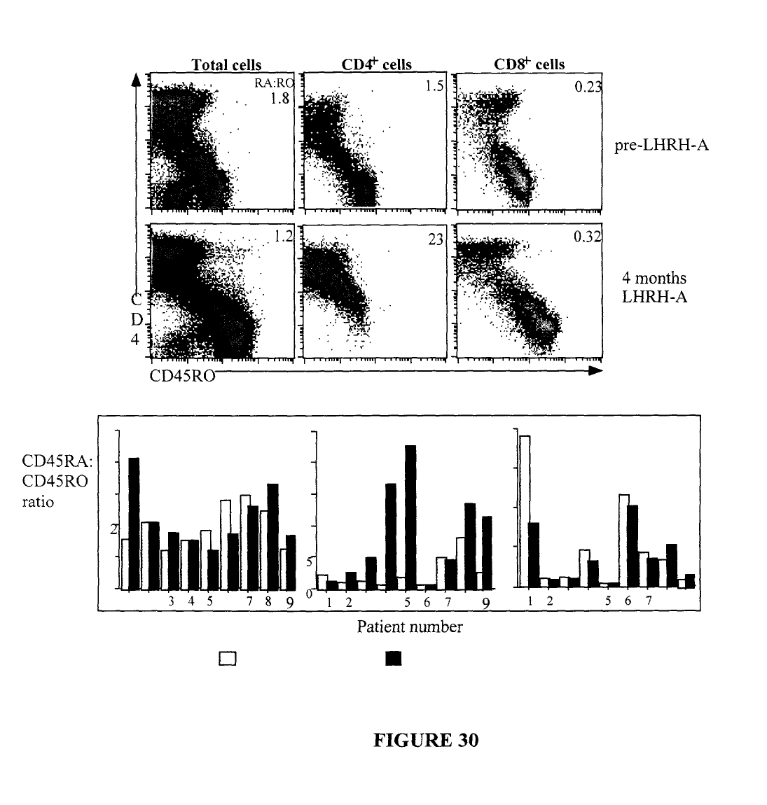

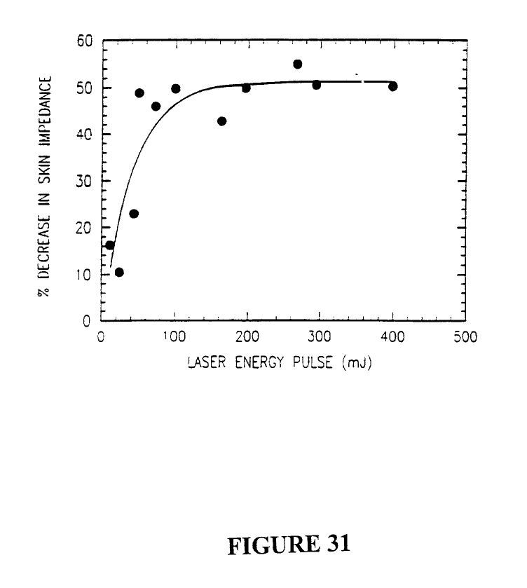

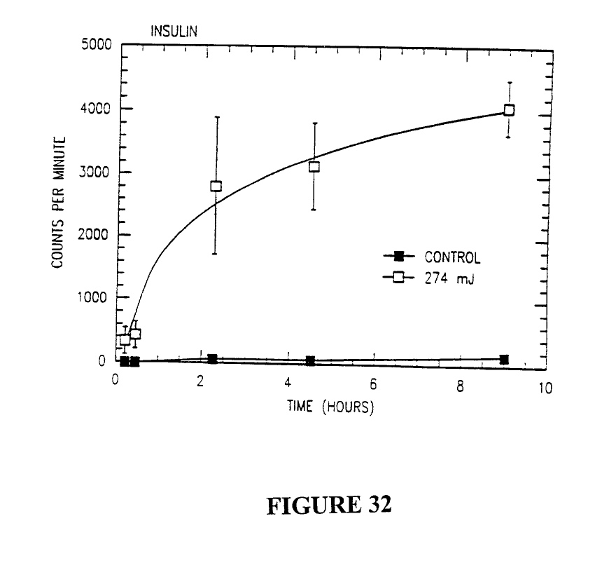

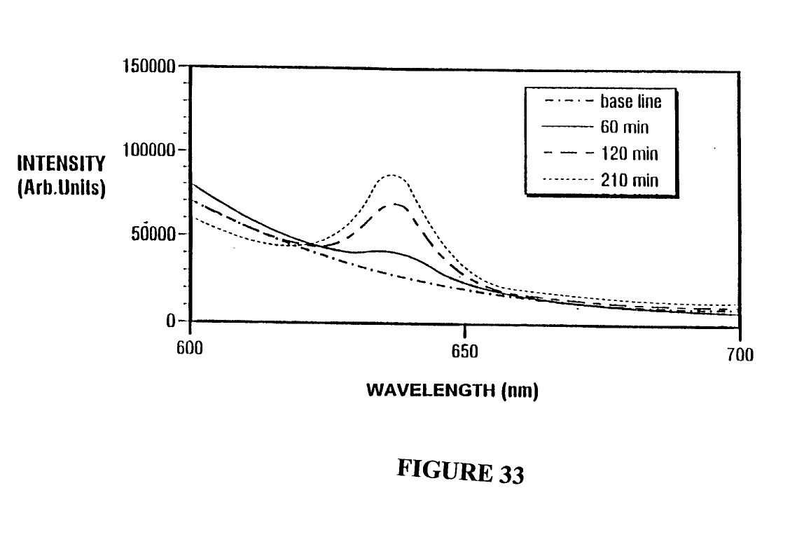

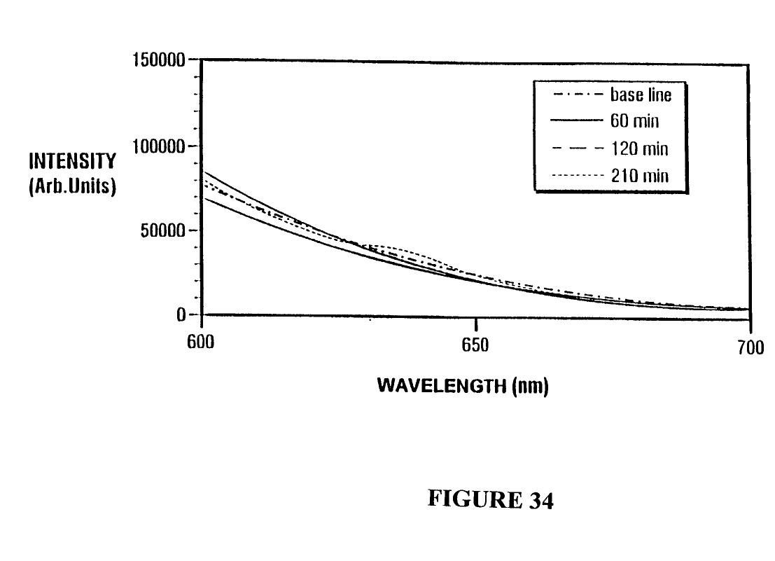

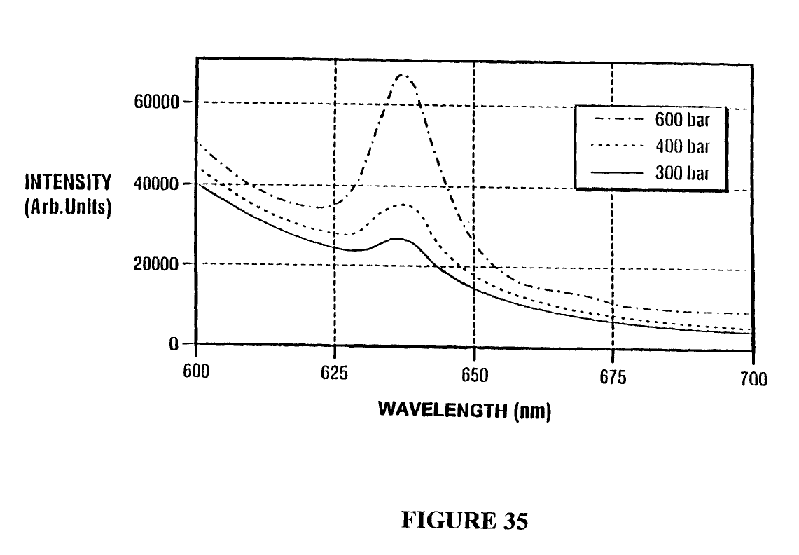

The major changes seen post-LHRH agonist treatment were within the T cell population of the peripheral blood. In particular there was a selective increase in the proportion of naïve (CD45RA⁺) CD4+ cells, with the ratio of naïve (CD45RA⁺) to memory (CD45RO⁺) in the CD4⁺ T cell subset increasing in 6/9 patients (FIG. 27).

### Conclusion

Thus it can be concluded that LHRH agonist treatment of an animal such as a human having an atrophied thymus can induce regeneration of the thymus. A general improvement has been shown in the status of blood T lymphocytes in these prostate cancer patients who have received sex-steroid ablation therapy. While it is very difficult to precisely determine whether such cells are only derived from the thymus, this would be very much the logical conclusion as no other source of mainstream (CD8 I [symbol] chain) T cells has been described. Gastrointestinal tract T cells are predominantly TCR KΛ or CD8 II chain.

### References

Aspinall, R., 1997, “Age-associated thymic atrophy in the mouse is due to adeficiency affecting rearrangement of the TCR during intrathymic T cell development,” J. Immunol. 158:3037.

Bahnson, A. B., et al., 1997, “Method for Retrovirus-Mediated Gene Transfer to CD34⁺-Enriched Cells,” in GENE THERAPY PROTOCOLS (P. D. Robbins, ed.), Humana Press, pp.249-263.

Bauer, G., et al., 1997, “Inhibition of Human Immunodeficiency Virus-1 (HIV-1) Replication After Transduction of Granulocyte Colony-Stimulating Factor-Mobilized CD34+ Cells From HIV-1-Infected Donors Using Retroviral Vectors Containing Anti-HIV-1 Genes,” Blood 89:2259-2267.

Belmont, J. W. and R. Jurecic, 1997, “Methods for Efficient Retrovirus-Mediated Gene Transfer to Mouse Hematopoietic Stem Cells,” in GENE THERAPY PROTOCOLS (P. D. Robbins, ed.), Humana Press, pp.223-240.

Berzins, S. P., Boyd, R. L. and Miller, J. F. A. P., 1998, “The role of the thymus and recent thymic migrants in the maintenance of the adult peripheral lymphocyte pool,” J. Exp. Med. 187:1839.

Bonyhadi, M. L., et al., 1997, “RevM10-Expressing T Cells Derived In Vivo From Transduced Human Hematopoietic Stem-Progenitor Cells Inhibit Human Immunodeficiency Virus Replication,” J. Virology 71:4707-4716.

Boyd, R. L., Tucek, C. L., Godfrey, D. I., Wilson, T. J, Davidson, N. J., Bean, A. G. D., Ladyman, H. M., Ritter, M. A. and Hugo, P., 1993, “The thymic microenvironment,” Immunology Today 14:445.

Bruijntjes, J. P., Kuper, C. J., Robinson, J. E. and Schutirman, H. J., 1993, “Epithelium-free area in the thymic cortex of rats,” Dev. Immunol. 3:113.

Capecchi, M. R., 1980, “High Efficiency Transformation by Direct Microinjection of DNA Into Cultured Mammalian Cells,” Cell 22:479-488.

Carayon, P., and Bord, A., 1992, “Identification of DNA-replicating lymphocyte subsets using a new method to label the bromo-deoxyuridine incorporated into the DNA,” J. Imm. Methods 147:225.

Douek, D. C., McFarland, R. D., Keiser, P. H., Gage, E. A., Massey, J. M., Haynes, B. F., Polis, M. A., Haase, A. T., Feinberg, M. B., Sullivan, J. L., Jamieson, B. D., Zack, J. A., Picker, L. J. and Koup, R. A., 1998, “Changes in thymic function with age and during the treatment of HIV infection,” Nature 396:690.

Fredrickson, G. G. and Basch, R. S., 1994, “Early thymic regeneration after irradiation,” Development and Comparative Immunology 18:251.

George, A. J. and Ritter, M. A., 1996, “Thymic involution with ageing: obsolescence or good housekeeping?,” Immunol. Today 17:267.

Godfrey, D. I, Izon, D. J., Tucek, C. L., Wilson, T. J. and Boyd, R. L., 1990, “The phenotypic heterogeneity of mouse thymic stromal cells,” Immunol. 70:66.

Godfrey, D. I, and Zlotnik, A., 1993, “Control points in early T-cell development,” Immunol. Today 14:547.

Graham, F. L. and Van Der Eb, A. J., 1973, “A New Technique for the Assay of Infectivity of Human Adenovirus 5 DNA,” Virology 52:456-457.

Hirokawa, K., 1998, “Immunity and Ageing,” in PRINCIPLES AND PRACTICE OF GERIATRIC MEDICINE, (M. Pathy, ed.) John Wiley and Sons Ltd.

Hirokawa, K. and Makinodan, T., 1975, “Thymic involution: the effect on T cell differentiation,” J. Immunol. 114:1659.

Hirokawa, K., Utsuyama M., Kasai, M., Kurashima, C., Ishijima, S. and Zeng, Y. -X., 1994, “Understanding the mechanism of the age-change of thymic function to promote T cell differentiation,” Immunology Letters 40:269.

Hobbs, M. V., Weigle, W. O., Noonan, D. J., Torbett, B. E., McEvilly, R. J., Koch, R. J., Cardenas, G. J. and Ernst, D. N., 1993, “Patterns of cytokine gene expression by CD4+ T cells from young and old mice,” J. Immunol. 150:3602.

Homo-Delarche, R. and Dardenne, M., 1991, “The neuroendocrine-immune axis,” Seminars in Immunopathology.

Huiskamp, R., Davids, J. A. G. and Vos, O., 1983, “Short- and long- term effects of whole body irradiation with fission neutrons or x-rays on the thymus in CBA mice,” Radiation Research 95:370.

Kendall, M. D., 1988, “Anatomical and physiological factors influencing the thymic microenvironment,” in THYMUS UPDATE I, Vol. 1. (M. D. Kendall, and M. A. Ritter, eds.) Harwood Academic Publishers, p. 27.

Kohn, D. B., et al., 1999, “A Clinical Trial of Retroviral-Mediated Transfer of a rev-Responsive Element Decoy Gene Into CD34⁺ Cells From the Bone Marrow of Human Immunodeficiency Virus-1 Infected Children,” Blood 94:368-371.

Kurashima, C, Utsuyama, M., Kasai, M., Ishijima, S. A., Konno, A. and Hirokawa, A., 1995, “The role of thymus in the aging of Th cell subpopulations and age-associated alteration of cytokine production by these cells,” Int. Immunol. 7:97.

Mackall, C. L. et. al., 1995, “Age, thymopoiesis and CD4+ T-lymphocyte regeneration after intensive chemotherapy,” New England J. Med. 332:143.

Mackall, C. L. and Gress, R. E., 1997, “Thymic aging and T-cell regeneration,” Immunol. Rev. 160:91.

Nabel, E. G., et al., 1992, “Gene Transfer In Vivo With DNA-Liposome Complexes: Lack of Autoimmunity and Gonadal Localization,” Hum. Gene Ther. 3:649-656.

Panoskaltsis, N, and C. N. Abboud, 1999, “Human Immunodeficiency Virus and the Hematopoietic Repertoire: Implications For Gene Therapy,” Frontiers in Bioscience 4:457.

Penit, C. and Ezine, S., 1989, “Cell proliferation and thymocyte subset reconstitution in sublethally irradiated mice: compared kinetics of endogenous and intrathymically transferred progenitors,” Proc. Natl. Acad. Sci, U.S.A. 86:5547.

Penit, C., Lucas, B., Vasseur, F., Rieker, T. and Boyd, R. L., 1996, “Thymic medulla epithelial cells acquire specific markers by post-mitotic maturation,” Dev. Immunol. 5:25.

Plosker, G. L. and Brogden, R. N., 1994, “Leuprorelin. A review of its pharmacology and therapeutic use in prostatic cancer, endometriosis and other sex hormone-related disorders,” Drugs 48:930.

Potter, H., Weir, L., and Leder, P., 1984, “Enhancer-dependent expression of Human Kappa Immunoglobulin Genes Introduced Into Mouse pre-B Lymphocytes by Electroporation,” Proc. Natl. Acad. Sci. USA 81:7161-7165.

Randle-Barrett, E. S. and Boyd, R. L., 1994, “Thymic microenvironment and lymphoid responses to sublethal irradiation,” Dev. Immunol. 4:1.

Scollay, R. G., Butcher, E. C. and Weissman, I. L., 1980, “Thymus cell migration. Quantitative aspects of cellular traffic from the thymus to the periphery in mice,” Eur. J. Immunol. 10:210.

Shortman, K., Egerton, M., Spangrude, G. J. and Scollay, R., 1990, “The generation and fate of thymocytes,” Seminars in Immuno. 2:3.

Starzl, T. E., Demetris, A. J., Murase, N., Ricardi, C. and Truce, M., 1992, “Cell migration, chimerism, and graft acceptance,” Lancet 339:1579.

Suda, T., and Zlotnik, A., 1991, “IL-7 maintains the T cell precursor potential of CD3-CD4-CD8-thymocytes,” J. Immunol. 146:3068.

Timm, J. A. and Thoman, M. L., 1999, “Maturation of CD4+ lymphocytes in the aged microenviroment results in a memory-enriched population,” J. Immunol. 162:711.

Thomson, A. W. and Lu, L., 1999, “Are dendritic cells the key to liver transplant?,” Immunology Today 20:20.

Tosi, R., Kraft, R., Luzi, P., Cintorino, M., Fankhause, G., Hess, M. W. and Cottier, H., 1982, “Involution pattern of the human thymus. 1. Size of the cortical area as a function of age,” Clin. Exp. Immunol. 47:497.

van Ewijk, W., Rouse, R. V. and Weissman, I. L., 1980, “Distribution of H-2 microenvironments in the mouse thymus,” J. Histochem. Cytochem. 28:1089.

Vickery, B. H., et al., eds., 1984, LHRH AND ITS ANALOGS: CONTRACEPTIVE & THERAPEUTIC APPLICATIONS, MTP Press Ltd., Lancaster, Pa.

von Freeden-Jeffry, U., Vieira, P., Lucian, L. A., McNeil, T., Burdach, E. G. and Murray, R., 1995, “Lymphopenia in interleukin (IL)-7 gene-deleted mice identifies IL-7 as a nonredundant cytokine,” J. Exp. Med. 181:1519.

Wiles, M. V., Ruiz, P. and Imhof, B. A., 1992, “Interleukin-7 expression during mouse thymus development,” Eur. J. Immunol. 22:1037.

Yang, N. -S. and P. Ziegelhoffer, 1994, “The Particle Bombardment System for Mammalian Gene Transfer,” In PARTICLE BOMBARDMENT TECHNOLOGY FOR GENE TRANSFER (Yang, N. -S. and Christou, P., eds.), Oxford University Press, New York, pp. 117-141.

Zlotnik, A. and Moore, T. A., 1995,“Cytokine production and requirements during T-cell development,” Curr. Opin. Immunol. 7:206.

1. A method for treating autoimmune disease in a patient comprising the steps of T cell ablation and reactivation of the thymus.

2. The method of claim 1 wherein the patient's thymus has been at least in part deactivated.

3. The method of claim 2 wherein the patient is post-pubertal.

4. The method of claim 1 further comprising the step of administering hematopoietic stem cells to the patient.

5. The method of claim 4 wherein the hematopoietic stem cells are CD34+.

6. The method of claim 4 wherein the hematopoietic stem cells are autologous.

7. The method of claim 4 wherein the hematopoietic stem cells are not autologous.

8. The method of claim 4 wherein the hematopoietic stem cells are administered about the time when the thymus begins to regenerate or shortly thereafter.

9. The method of claim 4 wherein the hematopoietic stem cells are provided at the time disruption of sex steroid mediated signaling to the thymus is begun.

10. The method of claim 1 wherein the method of disrupting the sex steroid mediated signaling to the thymus is through surgical castration to remove the patient's gonads.

11. The method of claim 1 wherein the method of disrupting the sex steroid mediated signaling to the thymus is through administration of one or more pharmaceuticals.

12. The method of claim 11 wherein the pharmaceuticals are selected from the group consisting of LHRH agonists, LHRH antagonists, anti-LHRH vaccines and combinations thereof.

13. The method of claim 12 wherein the LHRH agonists are selected from the group consisting of Eulexin, Goserelin, Leuprolide, Dioxalan derivatives, Triptorelin, Meterelin, Buserelin, Histrelin, Nafarelin, Lutrelin, Leuprorelin and Deslorelin.

14. The method of claim 1 wherein the auto-immune disease is alleviated.

15. A method for treating an allergy in a patient comprising the steps of T cell ablation and reactivation of the thymus.

16. The method of claim 15 wherein the patient's thymus has been at least in part deactivated.

17. The method of claim 15 wherein the patient is post-pubertal.

18. The method of claim 15 further comprising the step of administering hematopoietic stem cells to the patient.

19. The method of claim 18 wherein the hematopoietic stem cells are CD34+.

20. The method of claim 18 wherein the hematopoietic stem cells are autologous.

21. The method of claim 18 wherein the hematopoietic stem cells are not autologous.

22. The method of claim 18 wherein the hematopoietic stem cells are administered about the time when the thymus begins to regenerate or shortly thereafter.

23. The method of claim 18 wherein the hematopoietic stem cells are provided at the time disruption of sex steroid mediated signaling to the thymus is begun.

24. The method of claim 15 wherein the method of disrupting the sex steroid mediated signaling to the thymus is through surgical castration to remove the patient's gonads.

25. The method of claim 15 wherein the method of disrupting the sex steroid mediated signaling to the thymus is through administration of one or more pharmaceuticals.

26. The method of claim 25 wherein the pharmaceuticals are selected from the group consisting of LHRH agonists, LHRH antagonists, anti-LHRH vaccines and combinations thereof.

27. The method of claim 26 wherein the LHRH agonists are selected from the group consisting of Eulexin, Goserelin, Leuprolide, Dioxalan derivatives, Triptorelin, Meterelin, Buserelin, Histrelin, Nafarelin, Lutrelin, Leuprorelin and Deslorelin.

28. The method of claim 15 wherein the allergy is alleviated.
